(19) United States
(12) Patent Application Publication
(10) Pub. No.: US 2014/0303045 A1
(43) Pub. Date: Oct. 9, 2014

(54) Biocidal Systems and Methods of Use

(71) Applicant: Kemira Oyj, Helsinki (FI)

(72) Inventors: Scott Campbell, Conroe, TX (US); Angela Marie Johnson, Houston, TX (US)

(73) Assignee: Kemira Oyj, Helsinki (FI)

(21) Appl. No.: 14/245,825

(22) Filed: Apr. 4, 2014

Related U.S. Application Data
(60) Provisional application No. 61/808,489, filed on Apr. 4, 2013.

(51) Int. Cl. C09K 8/035; A01N 57/20; A01N 33/12; A01N 35/02; A01N 43/80; C09K 8/60; A01N 43/88

(52) U.S. Cl. CPC C09K 8/035; C09K 8/605; A01N 57/20; A01N 43/88; A01N 35/02; A01N 43/80; A01N 33/12
USPC 507/128; 507/130; 507/237; 507/240; 507/243; 514/126; 514/223.8

(57) ABSTRACT

Systems and methods for controlling microbial growth and/or activity in a gas field fluid or oil field fluid are provided, comprising: a) adding a first biocide component to the gas field fluid or oil field fluid in an amount effective to control microbial growth and/or activity; and b) after a delay, adding a second biocide component to the gas field fluid or oil field fluid.

### CROSS REFERENCE TO RELATED APPLICATIONS

This application claims the benefit of U.S. Provisional Application No. 61/808,489, filed on Apr. 4, 2013, which is incorporated herein by reference in its entirety.

### FIELD OF THE ART

The present disclosure relates to systems and methods for treating fluids with biocides to control microbial growth or activity, as well as fluids treated with the same.

### BACKGROUND

In the oil and gas industry, the development and operation of the oil field and gas field go through several distinct phases, all of which can be affected by unwanted microbial growth or activity. Microbial contamination may occur during drilling of the well, preparing the well for production, i.e. stimulation, and production itself.

It is desirable for the efficiency and success of any oil or natural gas production operation to protect water-based fluids from microbial contamination. After a well is drilled into a subterranean geological formation that contains oil, natural gas, and water, every effort is made to maximize the production of the oil and/or gas. To increase the permeability and flow of the oil and/or gas to the surface, the drilled wells are often subjected to well stimulation. Well stimulation generally refers to several post drilling processes used to clean the wellbore, enlarge channels, and increase pore space in the interval to be injected thus making it possible for fluids to move more readily into and out of the formation. In addition, typical reservoir enhancement processes such as waterflood and/or chemical-flood need to utilize biocide as part of the waterflood and/or chemical-flood package.

A typical well or field treatment process generally includes pumping specially engineered fluids at high pressure and rate into the subterranean geological formation. The high-pressure fluid (usually water containing specialty high viscosity fluid additives) exceeds the rock strength and opens a fracture in the formation, which can extend out into the geological formation for as much as several hundred feet. Certain commonly used fracturing treatments generally comprise a carrier fluid (usually water or brine) and a polymer, which is also commonly referred to as a friction reducer. Many well stimulation fluids will further comprise a proppant. Other compositions used as fracturing fluids include water with additives, viscoelastic surfactant gels, gelled oils, crosslinkers, oxygen scavengers, and the like.

The well treatment fluid can be prepared by blending the polymer with a fluid, such as an aqueous fluid. The purpose of the polymer is generally to increase the viscosity of the fracturing fluid; and to thicken the aqueous fluid so that solid particles of proppant can be suspended in the fluid for delivery into the fracture.

The well treatment fluids are subjected to an environment conducive to microbial growth and oxidative degradation. Microbial growth on polymers and other components of such fluids can materially alter the physical characteristics of the fluids. For example, microbial activity can degrade the polymer, leading to loss of viscosity and subsequent ineffectiveness of the fluids. Fluids that are especially susceptible to microbial degradation are those that contain polysaccharide and/or synthetic polymers such as polyacrylamides, polyglycosans, carboxyalkyl ethers, and the like. In addition to microbial degradation, these polymers are susceptible to oxidative degradation in the presence of free oxygen. The degradation can be directly caused by free oxygen or mediated by microorganisms. Thus, for example, polyacrylamides are known to degrade to smaller molecular fragments in the presence of free oxygen. Because of this, biocides and oxygen scavengers are frequently added to the well treatment fluid to control microbial growth or activity and oxygen degradation, respectively. The oxygen scavengers are generally derived from bisulfite salts.

Desirably, the biocide is selected to have minimal or no interaction with any of the components in the well stimulation fluid. For example, the biocide should not affect fluid viscosity to any significant extent and should not affect the performance of oxygen scavengers contained within the fluid. Other desirable properties for the biocide may include: (a) cost effectiveness, e.g., cost per liter, cost per cubic meter treated, and cost per year; (b) safety, e.g., personnel risk assessment (for instance, toxic gases or physical contact), neutralization requirements, registration, discharge to environment, and persistence; (c) compatibility with system fluids, e.g., solubility, partition coefficient, pH, presence of hydrogen sulfide in reservoir or formation, temperature, hardness, presence of metal ions or sulfates, level of total dissolved solids; (d) compatibility with other treatment chemicals, e.g., corrosion inhibitors, scale inhibitors, demulsifiers, water clarifiers, well stimulation chemicals, and polymers; and (e) handling, e.g., corrosiveness to metals and elastomers, freeze point, thermal stability, and separation of components.

### SUMMARY

Disclosed herein is a method of treating a gas field fluid or oil field fluid, comprising: a) adding a first biocide component to the gas field fluid or oil field fluid; and b) after a delay, adding a second biocide component in an amount effective to control microbial activity to the gas field fluid or oil field fluid; wherein the delay is at least about 1 minute wherein the first biocide and second biocide are added in an amount effective to control microbial activity.

Also disclosed herein is a method of treating a gas field fluid or oil field fluid, comprising: a) passing a gas field fluid or oil field fluid through a system; b) adding a first biocide component to the gas field fluid or oil field fluid via a first inlet to the system; and c) downstream from the first inlet, adding a second biocide to the gas field fluid or oil field fluid via a second inlet to the system.

The methods disclosed herein advantageously control microbial growth and/or activity in the fluid.

Also disclosed herein is a treated gas field fluid or oil field fluid comprising a first biocide component and a second biocide component, as well a system for treating gas field fluids and oil field fluids, comprising a first biocide component and a second biocide biocide component.

### BRIEF DESCRIPTION OF THE FIGURES

FIG. 1 is a graph which illustrates the effect of 3,5-dimethyl-1,3,5-thiadiazinane-2-thione and exemplary first biocide components on friction reduction performance of acrylamide-based polymer solutions.

FIG. 2 is a graph which illustrates the effect of several exemplary biocidal systems on friction reduction performance of acrylamide-based polymer solutions.

FIGS. 3 and 4 are graphs which illustrate the effect of exemplary biocidal systems on general heterotrophic bacteria (GHB) planktonic biocide efficacy with a delay of 5 minutes.

FIGS. 5 and 6 are graphs which illustrate the effect of exemplary biocidal systems on APB planktonic biocide efficacy with a delay of 5 minutes.

FIGS. 7 and 8 are graphs which illustrate the effect of exemplary biocidal systems on sulfur reducing bacteria (SRB) planktonic biocide efficacy with a delay of 5 minutes.

FIGS. 9 and 10 are graphs which illustrate the effect of exemplary biocidal systems on GHB (heterotrophic bacteria) planktonic biocide efficacy with a delay of 4 hours.

FIGS. 11 and 12 are graphs which illustrate the effect of exemplary biocidal systems on acid producing bacterial (APB) planktonic biocide efficacy with a delay of 4 hours.

FIGS. 13 and 14 are graphs which illustrate the effect of exemplary biocidal systems on SRB planktonic biocide efficacy with a delay of 4 hours.

FIGS. 15 and 16 are graphs which illustrate the effect of exemplary biocidal systems on GHB sessile biocide efficacy with a delay of 5 minutes.

FIGS. 17 and 18 are graphs which illustrate the effect of exemplary biocidal systems on APB sessile biocide efficacy with a delay of 5 minutes.

FIGS. 19 and 20 are graphs which illustrate the effect of exemplary biocidal systems on SRB sessile biocide efficacy with a delay of 5 minutes.

FIGS. 21 and 22 are graphs which illustrate the effect of exemplary biocidal systems on GHB sessile biocide efficacy with a delay of 4 hours.

FIGS. 23 and 24 are graphs which illustrate the effect of exemplary biocidal systems on APB sessile biocide efficacy with a delay of 4 hours.

FIGS. 25 and 26 are graphs which illustrate the effect of exemplary biocidal systems on SRB sessile biocide efficacy with a delay of 4 hours.

FIG. 27 is a graph which illustrates the effect of an exemplary biocidal system on SRB sessile biocide efficacy, in an active hydrofracing operation.

FIG. 28 is a graph which illustrates the effect of an exemplary biocidal system on APB sessile biocide efficacy, in an active hydrofracing operation.

### DETAILED DESCRIPTION

Described herein are biocidal systems, treated fluids and methods for controlling microbial growth and/or activity in a fluid.

The systems and methods disclosed herein are versatile and effective for use in gas field and oil field applications to control microbial growth and/or activity in fluids. The systems and methods described herein can be used to provide an enhanced antimicrobial activity, i.e., to control microbial viability or activity. In certain embodiments, the systems and methods can also be used to enhance the friction reduction capacity of friction reducing polymers, for example acrylamide-containing polymers.

As used herein, the term “control” refers to the ability of a component or composition or a method to influence microbial growth and/or activity in a treated fluid, e.g., to maintain a microbial population at or below a desired level for a desired period of time. This can vary from the complete prevention or inhibition of microbial growth and/or activity to partial inhibition or reduction of microbial growth or activity, and also includes maintaining a particular microbial population at a desired or acceptable level.

In exemplary embodiments, a biocidal system comprises a first biocide component and a second biocide component, wherein the second biocide component is 3,5-dimethyl-1,3,5-thiadiazinane-2-thione.

In exemplary embodiments, a method of treating a gas field fluid or oil field fluid comprises: a) adding a first biocide component to the gas field fluid or oil field fluid; and b) after a delay, adding a second biocide component to the gas field fluid or oil field fluid; wherein the delay is from at least about 1 minute, wherein the first biocide and second biocide are added in an amount effective to control microbial activity.

In exemplary embodiments, a method of treating a gas field fluid or oil field fluid comprises: a) passing a gas field fluid or oil field fluid through a system; b) adding a first biocide component to the gas field fluid or oil field fluid via a first inlet to the system; and c) downstream from the first inlet, adding a second biocide component to the gas field fluid or oil field fluid via a second inlet to the system wherein the first biocide and second biocide are added in an amount effective to control microbial activity.

In exemplary embodiments, a treated fluid comprises a first biocide component and a second biocide component, such as 3,5-dimethyl-1,3,5-thiadiazinane-2-thione.

Fluid

The exemplary embodiments provide biocidal systems, treated fluids and methods for controlling microbial growth and/or activity in a fluid. The fluid may be any fluid conducive to microbial contamination. In exemplary embodiments, the fluid has or is at risk of having an undesirable bio-burden.

In exemplary embodiment, the fluid is a gas field fluid or oil field fluid. In a particular embodiment, the fluid is a stimulation fluid, squeeze fluid, fracturing fluid, drilling mud, workover or completion fluid, hydrotest fluid, water injection or fluid injection for reservoir maintenance and Enhanced Oil Recovery (EOR).

In exemplary embodiments, the fluid comprises water and a polymer. In exemplary embodiments, the polymer may be any polymer used in a gas or oil field treatment fluid, for example a friction reducing polymer. In exemplary embodiments, the polymer comprises a polysaccharide, such as a galactomannan polymer, e.g. guar gum, a derivatized galactomannan polymer, starch, xanthan gum, a derivatized cellulose, e.g. hydroxycellulose or hydroxyalkyl cellulose; a polyvinyl alcohol polymer; or a synthetic polymer that is the product of a polymerization reaction comprising one or more monomers selected from the group consisting of vinyl pyrrolidone, 2-acrylamido-2-methylpropanesulfonic acid, acrylic acid, methacrylic acid, styrene sulfonic acid, acrylamide, and other monomers currently used for oil well treatment polymers. In exemplary embodiments, the polymer is water-soluble. Exemplary polymers include acrylamide-based polymers, hydrolyzed polyacrylamide, guar gum, hydroxypropyl guar gum, carboxymethyl guar gum, carboxymethylhydroxypropyl guar gum, hydroxyethyl cellulose, carboxymethylhydroxyethyl cellulose, hydroxypropyl cellulose, copolymers of acrylic acid and/or acrylamide, xanthan, starches, and mixtures thereof, among others. In an exemplary embodiment, the polymer is a copolymer of acrylic acid and/or acrylamide.

In exemplary embodiments, the biocidal system controlsmicrobial growth and/or activity in a gas field fluid or oil field fluid. As used herein, the term “fluid” includes but is not limited to gas field fluids or oil field fluids. The phrases “gas field fluid” or “oil field fluid” include stimulation fluid, squeeze fluid, fracturing fluid, drilling mud, workover or completion fluid hydrotest fluid, water injection or fluid injection for reservoir maintenance or Enhanced Oil Recovery (EOR), hydraulic fracturing fluids or other like compositions. In exemplary embodiments, the gas field fluid or oil field fluid is an aqueous fluid or a fluid that comprises water. In exemplary embodiments, the hydraulic fracturing fluid is a hydraulic fracturing fluid from an unconventional gas reservoir. While the exemplary embodiments described herein are described with reference to gas field fluids or oil field fluids, it is understood that the embodiments may be used in one or more other applications, as necessary or desired.

First Biocide Component

The method of the exemplary embodiments involves treating a fluid by applying biocides to control microbial growth and/or activity. As used herein, the term “biocide” refers to a substance that can control growth or activity of a microorganism (e.g., a bacterium) by chemical or biological means.

In an exemplary embodiment, the first biocide component comprises a fast acting biocide that has the ability to control microbial growth and/or activity within a short period of time.

In exemplary embodiments, the first biocide component comprises one or more biocides. In one embodiment, the first biocide component does not comprise 3,5-dimethyl-1,3,5-thiadiazinane-2-thione.

In exemplary embodiments, the first biocide component comprises two biocides.

In exemplary embodiments, the first biocide component comprises glutaraldehyde. In exemplary embodiments, the first biocide component comprises C₁₂₋₁₆-alkyl dimethyl benzyl ammonium chloride (ADBAC quat).

In exemplary embodiments, the first biocide component comprises glutaraldehyde and ADBAC quat. In exemplary embodiments, when the first biocide component comprises glutaraldehyde and ADBAC quat, the glutaraldehyde and ADBAC quat can be dosed separately or simultaneously, including, for example as individual compositions or as a solution, blend or mixture. In exemplary embodiments, the first biocide component comprises an aqueous blend of glutaraldehyde and ADBAC quat, e.g. AQUCAR™ 714 Water Treatment Microbiocide (available from The Dow Chemical Company).

In exemplary embodiments, the first biocide component comprises tetrakis(hydroxymethyl) phosphonium sulfate (THPS).

In exemplary embodiments, the first biocide component comprises 2,2-dibromo-3-nitrilopropionamide (DBNPA). In exemplary embodiments, the DBNPA is in the form of a formulation or solution, for example, a formulation containing 5% DBNPA, such as AQUCAR™ DB-5 Water Treatment Microbiocide (available from The Dow Chemical Company).

In exemplary embodiments, the first biocide component comprises [1,2-ethanediylbis(oxy)]bismethanol, such as BODOXIN™ AE (available from Ashland).

In exemplary embodiments, the first biocide component comprises 5-chloro-2-methyl-4-isothiazolin-3-one. In exemplary embodiments, the 5-chloro-2-methyl-4-isothiazolin-3-one is in the form of a composition which is adsorbed on an inert solid or silica-based solid, for example X-CIDE® 207 (available from Baker Petrolite). In exemplary embodiments, the first biocide component comprises 5-chloro-2-methyl-4-isothiazolin-3-one, magnesium nitrate and crystalline silica, for example X-CIDE® 207 (available from Baker Petrolite). In exemplary embodiments, the first biocide component comprises chlorine dioxide.

In exemplary embodiments, the first biocide component is selected from the group consisting of glutaraldehyde, ADBAC quat, an aqueous blend of glutaraldehyde and ADBAC quat, THPS, DBNPA, [1,2-ethanediylbis(oxy)]bismethanol, 5-chloro-2-methyl-4-isothiazolin-3-one, chlorine dioxide, and mixtures thereof.

In exemplary embodiments the first biocide component is a composition that converts relatively quickly into alkyl isothiocyanate, such as methylisothiocyanate (MITC). In exemplary embodiments, the first biocide component comprises a dithiocarbamate salt in an acidic environment. In exemplary embodiments, the first biocide component is a salt of N-methyldithiocarbamic acid, such as sodium N-methyldithiocarbamate or potassium N-methyldithiocarbamate. In exemplary embodiments, the first biocide component is a salt of N,N-dimethyldithiocarbamic acid, such as sodium N,N-dimethyldithiocarbamate, potassium N,N-dimethyldithiocarbamate, or zinc N,N-dimethyldithiocarbamate. In exemplary embodiments, the first biocide component is a salt of ethylene-1,2-bisdithiocarbamic acid, such as disodium ethylene-1,2-bisdithiocarbamate, or zinc ethylenebisdithiocarbamate. In certain embodiments, the alkyl group is a straight chain or branched C₁-C₆ hydrocarbon, e.g., a methyl, ethyl, propyl, butyl, pentyl, hexyl hydrocarbon chain.

In exemplary embodiments, the first biocide component further comprises one or more additives.

In exemplary embodiments, the first biocide component further comprises a enhancer of biocidal activity.

Second Biocide Component

In exemplary embodiments, the second biocide component is different than the first biocide component.

In exemplary embodiments, the second biocide component is any biocide that has the ability to control microbial growth and/or activity over a sustained time period.

In exemplary embodiments, a sustained period of time is a period of time that enables the prolonged use or recirculation of the fluid, for example, about 1 week, 2 weeks, 3 weeks, 4 weeks/1 month, about 2 months, about 6 months, or up to 1 year or more.

In exemplary embodiments, a sustained time period is a period of time that enables the extended storage of field fluid, e.g., prior to re-use in the field, for example, about 1 week, about 2 weeks, about 3 weeks, about 4 weeks/1 month, 2 months, about 6 months, or up to 1 year or more.

In exemplary embodiments, the second biocide component is a composition that converts at a relatively slow rate into an alkyl isothiocyanate, such as MITC. In exemplary embodiments, the first biocide component comprises a dithiocarbamate salt in an acidic environment. In exemplary embodiments, the second biocide component is a salt of N-methyldithiocarbamic acid, such as sodium N-methyldithiocarbamate or potassium N-methyldithiocarbamate. In exemplary embodiments, the second biocide component is a salt of N,N-dimethyldithiocarbamic acid, such as sodium N,N-dimethyldithiocarbamate, potassium N,N-dimethyldithiocarbamate, or zinc N,N-dimethyldithiocarbamate. In exemplary embodiments, the second biocide component is a salt of ethylene-1,2-bisdithiocarbamic acid, such as disodium ethylene-1,2-bisdithiocarbamate, or zinc ethylenebisdithiocarbamate. In certain embodiments, the alkyl group is a straight chain or branched C₁-C₆ hydrocarbon, e.g., a methyl, ethyl, propyl, butyl, pentyl, hexyl hydrocarbon chain.

In exemplary embodiments, the second biocide component is 3,5-dimethyl-1,3,5-thiadiazinane-2-thione. In exemplary embodiments the second biocide component is 3,5-dimethyl-1,3,5-thiadiazinane-2-thione in an alkaline environment.

In exemplary embodiments, the second biocide component further comprises one or more additives.

In exemplary embodiments, the second biocide component further comprises a enhancer of biocidal activity.

Biocidal System

In exemplary embodiments, a biocidal system comprises a first biocide component and a second biocide component. In exemplary embodiments, the second biocide component comprises 3,5-dimethyl-1,3,5-thiadiazinane-2-thione.

In exemplary embodiments, the biocidal system and methods described herein can be used to treat fluids and thereby control microbiological growth and or activity, such as in gas field or oil field applications. In certain embodiments, the methods provide a synergistic end result such that the antimicrobial activity of the system is improved over the antimicrobial activity of either biocide used alone at the same total dosage. In exemplary embodiments, the biocidal system controls the activity of microbes in water-based fluid very soon after it is introduced into the fluid (fast kill), and also provides an extended long term microbial control or prevents microbial re-growth. In exemplary embodiments, the systems and methods can be used to control, any microbial growth and/or activity in a fluid (e.g., a gas field fluid or oil field fluid), for example planktonic or sessile microbial growth and/or activity. In exemplary embodiments, the systems and methods can be used to treat fluids and thereby provide for long term control downhole to prevent reservoir souring, corrosion and/or other losses due to microbial activity.

In exemplary embodiments, the systems and methods can be used to inhibit growth and/or activity of various bacterial types, including but not limited to aerobic and non-aerobic bacteria, sulfur reducing bacteria (SRB), acid producing bacteria (APB) and the like. Specific examples include, but are not limited to, pseudomonad species, bacillus species, enterobacter species, serratia species, clostridia species and the like.

In one embodiment, the system and method are useful to control growth and/or activity of general heterotrophic bacteria (GHB), e.g., in treated fluids.

In another embodiment, the system and method are useful to control growth and/or activity of general aerobic bacteria (GAB)), e.g., in treated fluids.

In exemplary embodiments, the biocidal system comprises: a first biocide component, and a second biocide component, wherein the second biocide component comprises 3,5-dimethyl-1,3,5-thiadiazinane-2-thione. This system may be used to treat gas field fluids or oil field fluids. The first biocide component and the second biocide component may be added to such fluids separately and sequentially, according to the embodiments described herein.

In exemplary embodiments, the first biocide component is incompatible with the second component, e.g., 3,5-dimethyl-1,3,5-thiadiazinane-2-thione. For instance, when glutaraldehyde is combined with 3,5-dimethyl-1,3,5-thiadiazinane-2-thione in a mutual composition, e.g. in a composition containing both biocides that does not include substantial amounts of the oil field fluid, the efficacy of each biocide is compromised. Without being bound by any particular theory, it is believed that when these biocides are combined, changes to the chemistries occur which may compromise the biocidal activity of each. For example, one theory is that the 3,5-dimethyl-1,3,5-thiadiazinane-2-thione may increase the pH and/or provide amine moieties, providing an environment conducive to cross-linking or polymerization of the glutaraldehyde. The resulting mixture may have reduced biocidal effectiveness, and/or may show signs of chemical incompatibility, such as yellowing or precipitation.

In exemplary embodiments, the system may comprise one or more additional biocides.

In exemplary embodiments, the weight ratio of the second biocide component to the first biocide component is in the range of about 15:1 to about 1:5, about 10:1 to about 1:3, about 5:1 to about 1:2, about 3:1 to about 1:2, about 2:1 to about 1:2, or about 1:1 to about 1:2. In particular embodiments, the weight ratio of the active amount of the second biocide component to the first biocide component is in the range of about 15:1 to about 1:5, about 10:1 to about 1:3, about 5:1 to about 1:2, about 3:1 to about 1:2, about 2:1 to about 1:2, or about 1:1 to about 1:2.

In exemplary embodiments the first biocide component and the second biocide component are provided as individual compositions forming in situ a biocidal composition. In exemplary embodiments, the first biocide component and the second biocide component are provided as individual compositions which are sequentially added to a gas field fluid or an oil field fluid after one or more specified delays so as to optimize or maximize the antimicrobial effects of the two biocides.

The term “specified delays” as used herein may be temporal delays or may be due to procedural delays, for example those associated with the conducting the method steps such as adding the first biocide component and the second biocide component.

Methods of Use

The exemplary embodiments include methods of treating fluids, such as gas field fluids or oil field fluids, with the biocide system described herein in order to control microbial growth and/or activity in such fluids.

In exemplary embodiments, a method of treating a fluid, such as a gas field fluid or oil field fluid, comprises: a) adding a first biocide component to the gas field fluid or oil field fluid; and b) after a delay, adding a second biocide component to the fluid, wherein the first biocide and second biocide are added in an amount effective to control microbial activity, and wherein the delay is at least about 1 minute or, more particularly, from about 1 minute to about 4 hours. In exemplary embodiments, second biocide component comprises 3,5-dimethyl-1,3,5-thiadiazinane-2-thione.

In exemplary embodiments, a method of treating a gas field fluid or oil field fluid comprises: a) adding a first biocide component to the gas field fluid or oil field fluid; and b) after a delay, adding the second biocide component to the gas field fluid or oil field fluid; wherein the delay is at least about 1 minute wherein the first biocide and second biocide are added in an amount effective to control microbial activity. In exemplary embodiments, the second biocide component comprises 3,5-dimethyl-1,3,5-thiadiazinane-2-thione.

In an exemplary method, the first biocide component and the second biocide component may be added to the fluid in any amount effective to control microbial growth and/or activity.

In exemplary embodiments, the combined or total concentration of the first biocide component and the second biocide component in the fluid is greater than about 5 ppm, about 10 ppm, about 25 ppm, about 50 ppm, about 75 ppm, about 100 ppm, about 125 ppm, about 150 ppm, about 500 ppm or about 1000 ppm. In an exemplary embodiment, the combined concentration of the second biocide component and the first biocide component in the fluid is in the range of about 5 ppm to about 2000 ppm, about 5 ppm to about 1000 ppm, about 25 ppm to about 800 ppm, about 50 ppm to about 600 ppm, about 75 ppm to about 500 ppm, about 300 ppm to about 500 ppm, or about 25 ppm to about 50 ppm. In exemplary embodiments, the concentration of the second biocide component in the fluid is at least about 5 ppm. In exemplary embodiments, the components of the biocidal system may be added in any amount sufficient to produce a necessary or desired effect.

In exemplary embodiments, the combined or total concentration is the combined or total concentration of the active ingredients or active portion of the first biocide component and the second biocide component. In exemplary embodiments, the combined or total concentration, as active ingredients, of the first biocide component and the second biocide component in the fluid is greater than about 5 ppm, about 10 ppm, about 25 ppm, about 50 ppm, about 75 ppm, about 100 ppm, about 125 ppm, about 150 ppm, about 500 ppm or about 1000 ppm. In an exemplary embodiment, the combined concentration, as active ingredients, of the second biocide component and the first biocide component in the fluid is in the range of about 5 ppm to about 2000 ppm, about 5 ppm to about 1000 ppm, about 25 ppm to about 800 ppm, about 50 ppm to about 600 ppm, about 75 ppm to about 500 ppm, about 300 ppm to about 500 ppm, or about 25 ppm to about 50 ppm. In exemplary embodiments, the concentration of the second biocide component in the fluid is at least about 5 ppm as active ingredient. In exemplary embodiments, the components of the biocidal system may be added in any amount sufficient to produce a necessary or desired effect.

In exemplary methods, the components of the biocidal system (the first biocide component and the second biocide component) are separately added to a fluid as individual compositions. In exemplary embodiments, any composition or form of the second biocide component and the first biocide component may be used to deliver the active form of the components to the fluid. For example, each component may be added directly or indirectly to the fluid, and each component may be in the form of an aqueous solution, dry form, emulsion, aqueous dispersion or any other liquid or solid form. Any composition comprising a component of the biocidal system may further comprise additives or diluents which do not adversely impact the component. In certain embodiments, the second biocide component is in dry form, for example a granular solid or fine powder. In certain embodiments, the second biocide component is in the form of an aqueous solution, for example a 24% active aqueous solution of 3,5-dimethyl-1,3,5-thiadiazinane-2-thione. In exemplary embodiments, a caustic-based formulation of the second biocide component is used. In exemplary embodiments, a pH-adjusted composition comprising the second biocide component may be used in the systems and methods according to the embodiments, wherein the pH of the composition has been adjusted to decrease or increase the pH of the composition containing the second biocide component with pH modifying agents. In exemplary embodiments, the pH of the composition comprising the second biocide component has been increased with pH modifying agents.

In exemplary embodiments, the components of the biocidal system are added sequentially to the fluid with a delay between the additions. In an exemplary embodiment, the second biocide component and the first biocide component are added to the fluid sequentially and the first biocide component is added first.

The delay between additions may be any amount of time as necessary or desired to achieve activity necessary or desired result. In exemplary embodiments, the delay between the addition of the first biocide component and the addition of the second biocide component is about 1 minute, about 2 minutes, about 3 minutes, about 4 minutes, about 5 minutes, about 6 minutes, about 7 minutes, about 8 minutes, about 9 minutes, about 10 minutes, about 15 minutes, about 20 minutes, about 25 minutes, about 30 minutes, about 1 hour, about 2 hours, about 4 hours, or about 24 hours. In exemplary embodiments, the delay is from about 1 minute to about 24 hours, about 1 minute to about 4 hours, about 1 minute to about 2 hours, about 1 minute to about 1 hour, about 1 minute to about 30 minutes, about 1 minute to about 25 minutes, about 1 minute to about 20 minutes, about 5 minutes to about 15 minutes, about 5 minutes to about 12 minutes, about 5 minutes to about 10 minutes, or about 1 minute to about 10 minutes.

In exemplary embodiments, the delay for a method may be determined based on a variety of factors, including, for example, the desired level or profile (e.g., activity over time) of antimicrobial activity, the dissolution rate of the biocide components in the fluid, the nature of the biocide components, the half-lives of the biocides, and the structure and/or operating conditions of the fluidic system.

In exemplary embodiments, one or more of the first biocidal component and second biocide component of the biocidal system may be added in multiple doses. For example, one or both of the second biocide component and/or the first biocide component may be added in a single dose or in multiple doses to a pipeline, reservoir or other part of a fluidic system.

In one embodiment, a method of controlling microbial growth and/or activity in a fluid, such as a gas field fluid or oil fuel fluid, comprises a) adding a first biocide component to the fluid; and b) after a delay, adding a second biocide component to the fluid; wherein the delay is at least about 1 minute wherein the first biocide and second biocide are added in an amount effective to control microbial activity. In exemplary embodiments, the first and second biocide components are different. In certain embodiment, the second biocide component is 3,5-dimethyl-1,3,5-thiadiazinane-2-thione. In certain embodiments, the first biocide component is not 3,5-dimethyl-1,3,5-thiadiazinane-2-thione.

In another embodiment, a method of controlling microbial growth in a fluid, such as a gas field fluid or oil field fluid, comprises: a) passing a fluid through a system; b) adding a first biocide component to the fluid via a first inlet to the system; and c) downstream from the first inlet, adding a second biocide component to the fluid via a second inlet to the system wherein the first biocide and second biocide are added in an amount effective to control microbial activity. In exemplary embodiments, the first and second biocide components are different. In certain embodiment, the second biocide component is 3,5-dimethyl-1,3,5-thiadiazinane-2-thione. In certain embodiments, the first biocide component is not 3,5-dimethyl-1,3,5-thiadiazinane-2-thione.

In an exemplary embodiment, a method of treating a gas field fluid or oil field fluid, comprises: a) passing a gas field fluid or oil field fluid through a fluidic system; b) adding a first biocide component to the gas field fluid or oil field fluid via a first inlet to the fluidic system; and c) downstream from the first inlet, adding a second biocide to the gas field fluid or oil field fluid via a second inlet to the fluidic system wherein the first biocide and second biocide are added in an amount effective to control microbial activity.

In an exemplary embodiment, the fluidic system is any part of the system associated with a hydraulic fracturing process in which field fluid is circulated. An exemplary fluidic system for a hydraulic fracturing process provides, generally, a system for transporting one or more hydraulic fracturing fluids from a one more above ground locations, to one or more subterranean locations. An exemplary fluidic system may include a number of systems or processes including, inter alia, storage systems, supply systems, transport systems (e.g., pipes, valves, pumps), pressure control systems, blending or mixing systems, water treatment systems, and the like. In an exemplary embodiment, the first biocide component and second biocide component may be separately added (e.g., by injection) to the fluids in the fluidic system at any location in the system. For example, a biocide may be added to the fluidic system at one or more of the following locations: a frac pond, a mixing manifold upstream of a frac tank, a frac tank, an outlet manifold of a frac tank, a blender, a chemical injection pump such as one just upstream of the high pressure pump and downstream of the booster pump, or other locations. In exemplary embodiments, determination of the location of the addition/injection of a biocide component may depend on the desired effectiveness of a biocide component in the fluidic system. For example, determination of the location of the addition/injection location may take into consideration a number of variables, including, for example, the pH of the system, and residence time in the system. For example, in an exemplary embodiment, if a biocide is injected into the fluidic system after a frac tank, it may have a residence time within the fluidic system of less than about 10 minutes, or less than about 5 minutes. In an exemplary embodiment, if a biocide is injected into the fluidic system at a frac tank, the biocide may have a residence time of greater than about 24 hours. These and other factors may affect the activity of the biocide within the system.

In exemplary embodiments, the gas field fluid or oil field fluid may be a stimulation fluid, squeeze fluid, fracturing fluid, drilling mud, workover or completion fluid, hydrotest fluid, water injection or fluid injection for reservoir maintenance or Enhanced Oil Recovery (EOR).

In exemplary embodiments, a biocidal system comprises a second biocide component (e.g., 3,5-dimethyl-1,3,5-thiadiazinane-2-thione) and a first biocide component may be used in a gas field or oil field application. In exemplary embodiments, a biocidal system comprising 3,5-dimethyl-1,3,5-thiadiazinane-2-thione and a first biocide component may be used in a gas field fluid or oil field fluid. In exemplary embodiments, the gas field fluid or oil field fluid is a stimulation fluid, squeeze fluid, fracturing fluid, drilling mud, workover or completion fluid or hydrotest fluid. In exemplary embodiments, the biocidal system is used for inhibiting microbial growth or activity in a gas field fluid or oil field fluid.

In exemplary embodiments, the gas field fluid or oil field fluid comprises water, for example fresh water, saline water or recirculated water.

In exemplary embodiments, the gas field fluid or oil field fluid comprises water and a polymer. In exemplary embodiments, the polymer may be any polymer used in a gas or oil field treatment fluid, for example a friction reducing polymer. In exemplary embodiments, the polymer comprises a polysaccharide, such as a galactomannan polymer, e.g. guar gum, a derivatized galactomannan polymer, starch, xanthan gum, a derivatized cellulose, e.g. hydroxycellulose or hydroxyalkyl cellulose; a polyvinyl alcohol polymer; or a synthetic polymer that is the product of a polymerization reaction comprising one or more monomers selected from the group consisting of vinyl pyrrolidone, 2-acrylamido-2-methylpropanesulfonic acid, acrylic acid, methacrylic acid, styrene sulfonic acid, acrylamide, and other monomers currently used for oil well treatment polymers. In exemplary embodiments, the polymer is water-soluble. Exemplary polymers include acrylamide-based polymers, hydrolyzed polyacrylamide, guar gum, hydroxypropyl guar gum, carboxymethyl guar gum, carboxymethylhydroxypropyl guar gum, hydroxyethyl cellulose, carboxymethylhydroxyethyl cellulose, hydroxypropyl cellulose, copolymers of acrylic acid and/or acrylamide, xanthan, starches, and mixtures thereof, among others. In an exemplary embodiment, the polymer is a copolymer of acrylic acid and/or acrylamide.

In exemplary embodiments, the gas field fluid or oil field fluid can further comprise one or more additives. For example, an additive may be included to provide any necessary or desired characteristic, such as to enhance the stability of the fluid composition itself to prevent breakdown caused by exposure to oxygen, temperature change, trace metals, constituents of water added to the fluid composition, and/or to prevent non-optimal crosslinking reaction kinetics. Often, the choice of components used in fluid compositions is determined to a large extent by the properties of the hydrocarbon-bearing formation on which they are to be used. Exemplary additives include, but are not limited to, oils, salts (including organic salts), crosslinkers, polymers, biocides, corrosion inhibitors and dissolvers, enzymes, pH modifiers (e.g., acids and bases), breakers, metal chelators, metal complexors, antioxidants, oxygen scavengers, wetting agents, polymer stabilizers, clay stabilizers, scale inhibitors and dissolvers, wax inhibitors and dissolvers, asphaltene precipitation inhibitors, water flow inhibitors, fluid loss additives, chemical grouts, diverters, sand consolidation chemicals, proppants, permeability modifiers, viscoelastic fluids, gases (e.g., nitrogen and carbon dioxide), foaming agents, defoaming agents, and controlled-release vehicles.

In an exemplary embodiment, the biocidal system may be used in a well stimulation application. For example, a fluid containing the biocidal system can be injected directly into the wellbore to react with and/or dissolve substances affecting permeability; injected into the wellbore and into the formation to react with and/or dissolve small portions of the formation to create alternative flowpaths; or injected into the wellbore and into the formation at pressures effective to fracture the formation.

In an exemplary embodiment, the field fluid is a well injection composition. The well injection composition is not particularly limited, and can comprise an injection fluid for removing a production fluid such as oil from a subterranean formation. The injection fluid can be any fluid suitable for forcing the production fluid out of the subterranean formation and into a production wellbore where it can be recovered. For example, the injection fluid can comprise an aqueous fluid such as fresh water or salt water (i.e., water containing one or more salts dissolved therein), e.g., brine (i.e., saturated salt water) or seawater In an exemplary embodiment, the well injection composition can be used in a flooding operation (e.g., secondary flooding as opposed to a primary recovery operation which relies on natural forces to move the fluid) to recover a production fluid, e.g., oil, from a subterranean formation. The flooding operation entails displacing the well injection composition through an injection well (or wells) down to the subterranean formation to force or drive the production fluid from the subterranean formation to a production well (or wells). The flooding can be repeated to increase the amount of production fluid recovered from the reservoir. In subsequent flooding operations, the injection fluid can be replaced with a fluid that is miscible or partially miscible with the oil being recovered.

An exemplary injection well is not particularly limited and can include a cement sheath or column arranged in the annulus of a wellbore, wherein the annulus is disposed between the wall of the wellbore and a conduit such as a casing running through the wellbore. Thus, the well injection composition can pass down through the casing into the subterranean formation during flooding. The biocidal system in the well injection composition can serve to reduce microbial growth or activity on the cement sheath and the conduit therein without significantly affecting the materials with which it comes in contact, including the components of the well injection composition.

In exemplary embodiments, the methods can be used without significant changes to the pH of the fluid or the fluidic system to which the biocide system is applied, for example the pH of the fluid or fluidic system to which the biocide system is applied will change less than about 2 pH unit or less than about 1 pH unit. In exemplary embodiments, the methods can be used without substantially adversely impacting the friction reduction capacity of the friction reducing polymers in fluid or in the fluidic system to which the biocide system is applied. In exemplary embodiments, the addition of the biocidal system to a fluid or fluidic system containing friction reducing polymers will decrease the friction reduction capacity of the friction reducing polymers less than about 10%. In exemplary embodiments, the friction reduction capacity of a fluid or fluidic system to which the biocide system is applied is equal to or greater than the friction reduction capacity of a comparable fluid or fluidic system to which the second biocide component without additional biocides is applied.

The following examples are presented for illustrative purposes only, and are not intended to limit the scope of the invention.

### EXAMPLES

For the following Examples 1-3, several stock solutions of exemplary first biocides were prepared by adding following amounts of biocides to a 25 mL volumetric flask, and adding deionized water to fill the flask to the 25 mL mark.

| First Biocide | |
|---|---|
| Sample: | Preparation: |
| GLUT | 1.00 grams of commercially-available Glutaraldehyde. |
| THPS | 0.67 grams of commercially-available THPS |
| DBNPA | 1.25 grams of DBNPA 5% solution (AQUACAR ™ |
| | DB5, commercially available from The Dow Chemical |
| | Company, Midland, MI). |
| GLUT + ADBAC | 3.03 grams of a Glut, ADBAC quat blend |
| | (AQUACAR ™ 714, commercially available from |
| | The Dow Chemical Company, Midland, MI). |
| ADBAC quat | 1.00 g of a commercially-available ADBAC quat |
| Isothiazolinone | 6.67 grams of 5-chloro-2-methyl-4-isothiazolin-3-one |
| | adsorbed on an inert solid (X-CIDE ™ 207, |
| | commercially available from Baker Petrolite |
| | Corporation, Sugar Land, TX) |

A stock solution of the second biocide solution was prepared with 3,5-dimethyl-1,3,5-thiadiazinane-2-thione (AMA®-324, a caustic-based biocide commercially available from Kemira Chemicals, Inc., Atlanta, Ga.). The AMA®-324 stock solution was prepared by adding 2.08 grams of AMA®-324 to a 25 mL flask, and filling the flask to the 25 mL mark with deionized water.

### Example 1

### pH Assessment

In this example, the effect on pH was evaluated for polymer solutions having various biocidal systems.

Seven standard polymer solutions were prepared by preparing a commercially-available polyacrylamide polymer using standard methods. The polymer was added to artificial seawater formulation (approximately 27° C., with the pH adjusted to 6.5 and buffered accordingly) and the solution was mixed utilizing a magnetic stirrer to obtain 200 mL of a 300 ppm (active) polymer solution. After mixing, the initial pH of each polymer solution was measured and recorded. The pH was measured using a calibrated bench top pH meter.

After the initial pH reading, 1 mL of the AMA-324 stock solution was added to 200 mL of the polymer solution to provide 100 ppm (active) of the biocide, and mixed for 10 minutes. The pH of the solution with the AMA-324 was then measured and recorded.

Following pH measurement of the polymer solutions containing AMA-324, 1 mL of a first biocide component stock solution (as described in Table 1) was added to each polymer mixture. The concentration of the biocide stock solutions were prepared to provide 100 ppm (active) in the polymer solution, except for DBNPA which was evaluated at 50 ppm (active). The polymer solutions with the AMA-324 and the first biocide components were mixed for 30 minutes, after which the pH was again measured. The pH of the solution was adjusted as indicated in Table 1, and physical changes to the appearance of the solution were recorded (cloudiness, precipitation, flocculation, pH, etc.).

Finally, one control experiment was performed with only the addition of AMA-324 to the polymer solution, stirred for 40 minutes with no other biocide addition.

The pH values for each step of the protocol, and visual observations of the resultant solutions are presented in Table 1.

TABLE 1
pH changes and visual observations for compatibility of first biocide with AMA-324.

| First Biocide: | | GLUT + | Isothia- | | | ADBAC | (AMA- |
|---|---|---|---|---|---|---|---|
| Step: | THPS | ADBAC | zolinone | DBNPA** | GLUT | quat | 324 alone) |
| Starting pH (without | 6.29 | 6.21 | 6.19 | 6.14 | 6.08 | 6.71 | 6.29 |
| Biocide) | | | | | | | |
| after addition of AMA-324 | 9.04 | 9.25 | 9.1 | 9.2 | 9.2 | 9.64 | 9.3 |
| after addition of first | 6.8 | 9.17 | 9.08 | 8.04 | 9.29 | 9.57 | |
| biocide | | | | | | | |
| end | 7.13 | 9.33 | 7.82 | 8.44 | 9.12 | 9.18 | 9.32 |
| adjusted pH | | 6.78 | 7.08 | 7.12 | 7.37 | 6.33 | 7.41 |
| Observations on adjusted | clear | clear | clear, after | cloudy | clear | cloudy | clear |
| pH solution | | | filtering | | | | |
| | | | inert solid | | | | |

**repeated the procedure for AMA-324 + DBNPA and AMA-324 + ADBAC Quat using deionized water instead of polymer solution, and the solution still turned cloudy.

As shown in Table 1, the polymer solutions to which AMA-324 was added in combination with THPS, Glut/ADBAC, isothiazolinone, and Glut, were clear. In contrast, the solutions in which the AMA-324 was added in combination with DBNPA and ADBAC Quat has some turbidity. This result was repeated when the polymer solution was substituted with deionized water.

All solutions except AMA-324 with THPS required pH adjustment (lowering) to get into the test range.

The order of addition of the biocides or biocide components to the polymer solution is not of significance in this Example because the pH of the resulting solution will be the same regardless of the order of addition. A time allowance to account for the potential interaction between AMA-324 and the first biocide components was provided in these tests.

### Example 2

### Friction Reduction Assessment

Friction reduction measurements were performed on a friction meter, which pumps water or brine solutions through ¼″ tubing at a rate of 2.2 gallons per minute (Reynolds number=40,000).

An artificial seawater formulation was provided, at approximately 27° C., with the pH adjusted to 6.5 and buffered accordingly. Several biocide solutions were prepared by adding to the artificial seawater formulation a sufficient amount of one of the first biocide component stock solutions and/or AMA-324 stock solution to provide a solution having about 100 ppm (active) of the respective biocide, except for DBNPA which was evaluated at 50 ppm (active). Polymer solution samples were prepared by adding 4 L of a water (artificial seawater—control) or biocide solution to a 5 L beaker, mixing thoroughly with an overhead mechanical stirrer, adding 1 gram of a commercially-available emulsion polyacrylamide polymer to the beaker, and stirring the polymer solution for about 30 minutes.

A water baseline was established in the friction loop by adding 4 L of water (artificial seawater) to the friction meter funnel, and circulated until system stabilized and pressure recorded. Following the pressure reading, water was pumped out of the funnel and the loop system.

Each 4 L polymer-biocide solution (and control solution) was separately added to the friction loop. The pump was run and the gauge pressure was recorded following the stabilization of the system. Upon completion of the test, the polymer solution was pumped out of system and the system was cleaned with tap water prior to the next run.

The percent friction reduction was calculated. For each combination of gauge and time, the following formula was used and the data reported as % Friction Reduction:

$$\% FR = \frac{{P}_{0} - {P}_{t}}{{P}_{0}} \times 100$$

where P₀ is the baseline pressure, and Pt is the pressure for the polymer solution.

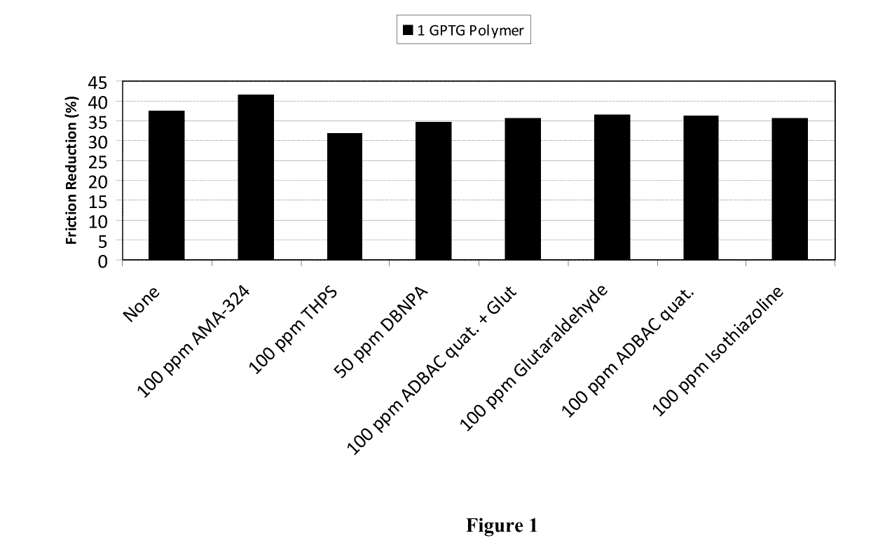

FIG. 1 shows the effect of several single-biocide treatments on the friction reduction performance. AMA-324 biocide evaluated as a single treatment improved the friction reducer's performance on 1 GPTG acrylamide-based polymer made down in water.

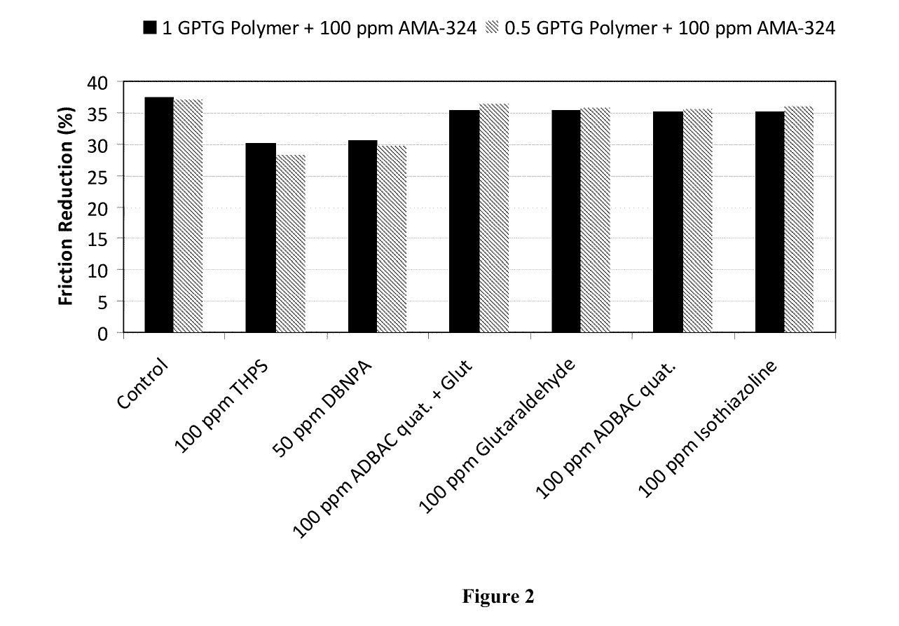

FIG. 2 shows the effects of exemplary dual biocide systems including a first biocide component and AMA-324. In FIG. 2, the control sample included the polymer but not AMA-324. Except samples including THPS and DBNPA, all other exemplary biocidal systems did not significantly affect (reduce) the performance of the friction reducing polymer.

### Example 3

### Biocide Efficacy Testing Against Planktonic Bacteria

In this example, the efficacy of the first biocide components and the efficacy of AMA-324 against planktonic bacteria (time kill dependent study) and sessile bacterial development was assessed by the following protocol.

Preparation of Planktonic Test Cultures

A water chemistry analysis was performed to establish water compatibility, carbon source and energy limitations that may exist for the bacteria that could negatively affect the results. One control and 14 test reactors were set-up. An environmental consortium of bacteria cultured from oilfield water injection system operating at an equivalency to system waters of standard seawater chemistry of approximately 2.5% TDS with a pH of 7.5 was used to inoculate a base culture stock of bacteria for biocide testing.

A base stock culture was created to prevent toxicity and/or bacterial transfer shock from potentially affecting results and data interpretation. The base stock culture consists of general aerobic bacteria (GHB), acid producing bacteria (APB), and sulfate reducing bacteria (SRB), and was created by inoculating 9 mL of fresh respective bacterial growth media with 1 mL of respective bacterial consortium. The newly inoculated stock cultures were then incubated at 35° C. for 2-4 days to revive the bacteria and promote the log phase of growth. Prior to inoculation, the bacterial cells were centrifuged and washed to remove as much sulfide as possible as well as residual media.

Preparation of Planktonic Biocide Test Suspensions

For this test, 14 bottles with artificial aerobic seawater with an adjusted pH of 6.5 buffered with HEPES buffer were set up including 1 control. These bottles were then inoculated with washed log phase bacterial cultures, such that a final bacterial population of approximately 1×106 of each GHB, APB, and SRB was achieved in the test bottles. This water solution with bacteria was then allowed to stabilize for 4 hours prior to adding any biocide. Prior to collecting the samples, the flask was mixed vigorously to ensure re-suspension and equal distribution of bacteria. Time 0 samples were collected at this time from all test reactors and the control. Following inoculation, stabilization, and sampling, biocide were added to the reactors excluding the control. Immediately following the additions of biocide, stainless steel corrosion coupons were placed in the bottom of the reactors. (A time 0 sample is swabbed from the sterile coupons to verify sterility of the coupons prior to inserting them into the test reactor.) No further mixing of the reactors was performed. Care was taken to avoid any agitation and/or mixing during transport and or sampling.

Biocide Addition

The AMA-324 and the first biocide components were added to all the reactors following the time 0 sample collection. The first biocide components were added to achieve the following active concentration:

| | Active Concentration |
|---|---|
| First Biocide Component | (v/v) |
| Glutaraldehyde | 100 |
| THPS | 100 |
| DBNPA | 100 |
| Glutaraldehyde and ADBAC quat | 100 |
| ADBAC quat | 100 |
| 5-chloro-2-methyl-4-isothiazolin-3-one | 50 |
| adsorbed on an inert solid | |

Of the 13 total reactors, 6 reactors were designated for the 5 min. test set and 6 were designated for the 4 hrs. test set.

For the 5-minute test set, 5 min. following the addition of the first biocide component, approximately 400 ppm of AMA-324 product was added to the 6 reactors and the reactors were placed in the incubator and all shaking was minimized or eliminated for the course of the study.

For the 4-hour test set, 4 hrs. following the initial addition of the first biocide component, the reactors were shaken and a 4 hr. sample was collected prior to adding the AMA-324 biocide. Immediately following the sampling, approximately 400 ppm of AMA-324 product was added to these 6 reactors delegated for 4 hrs., and the reactors were placed in an incubator and all shaking was minimized or eliminated for the course of the study.

Planktonic and Sessile Sampling

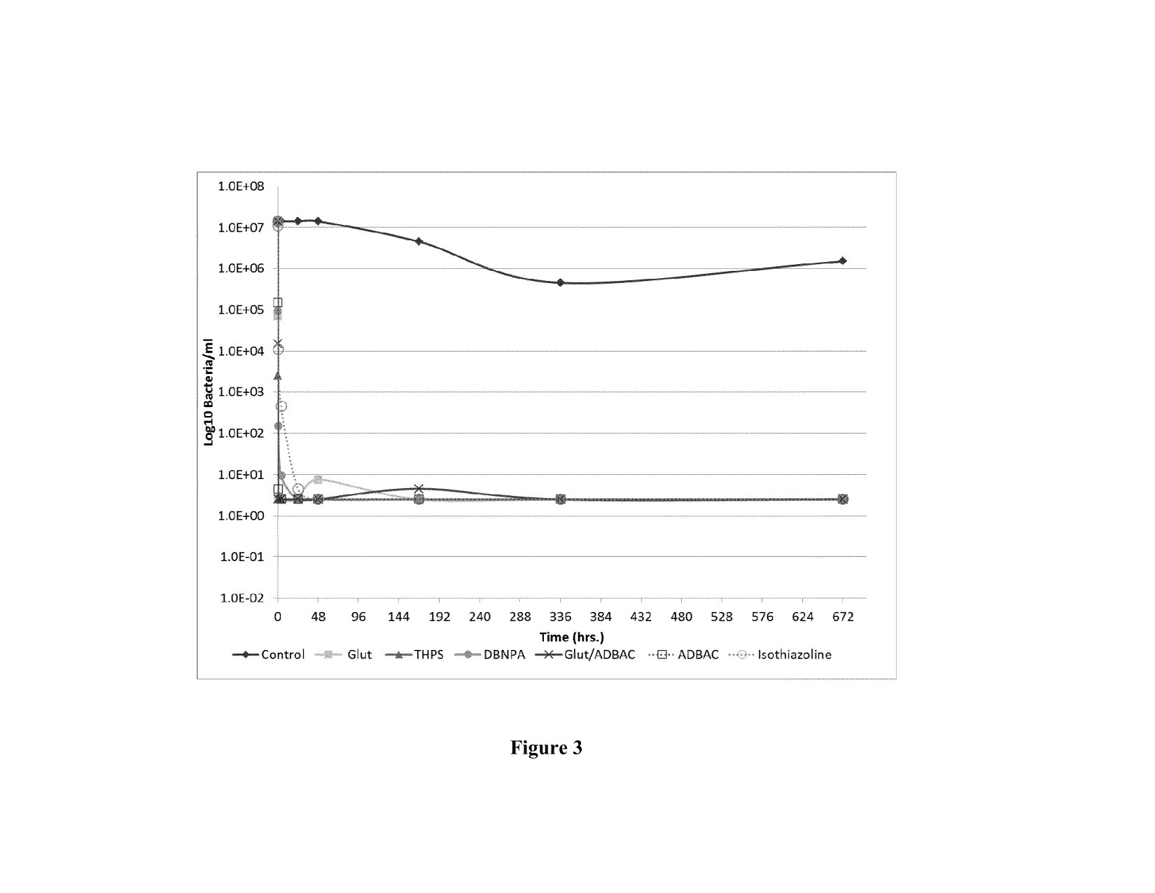

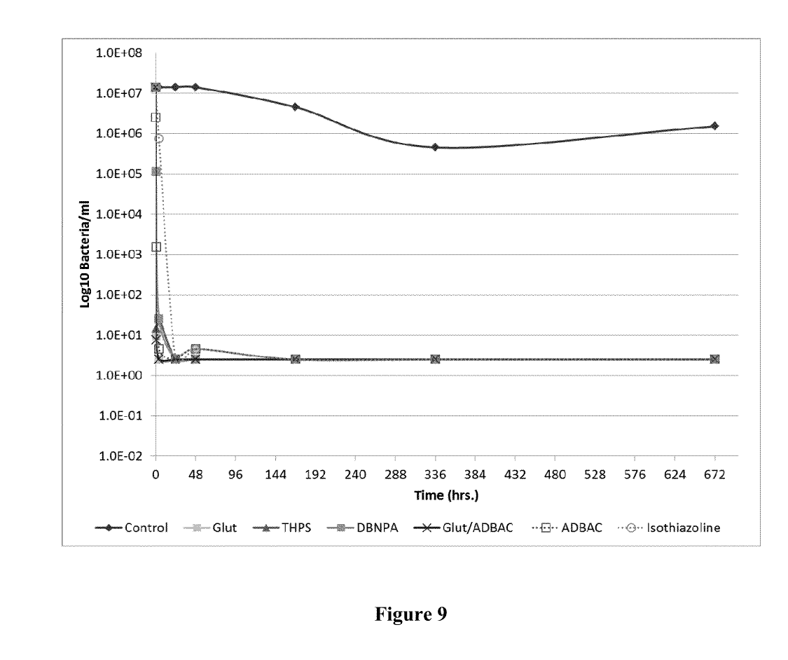

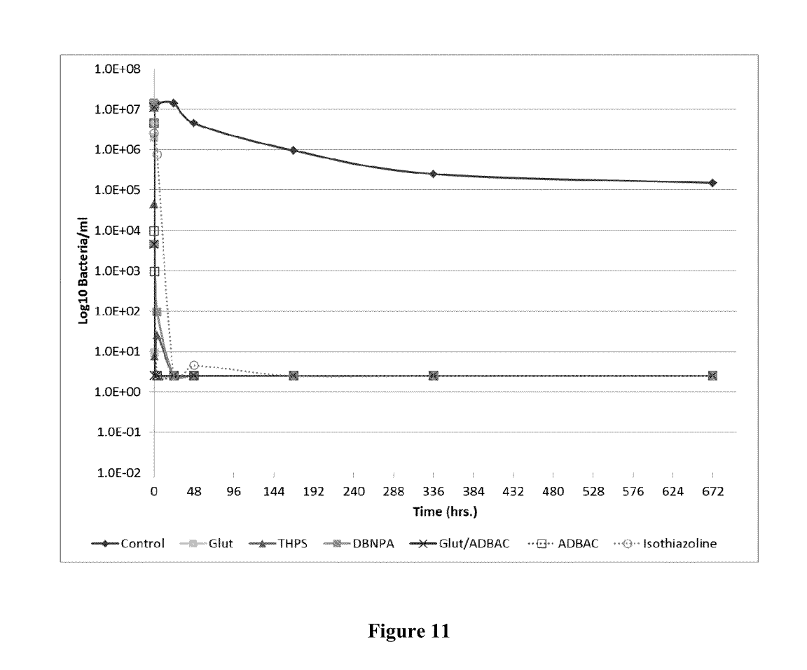

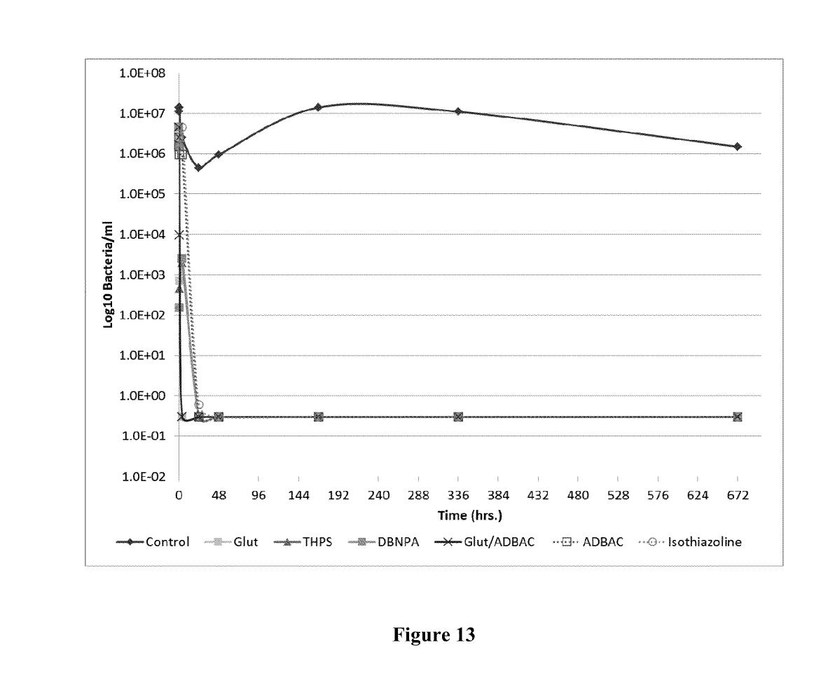

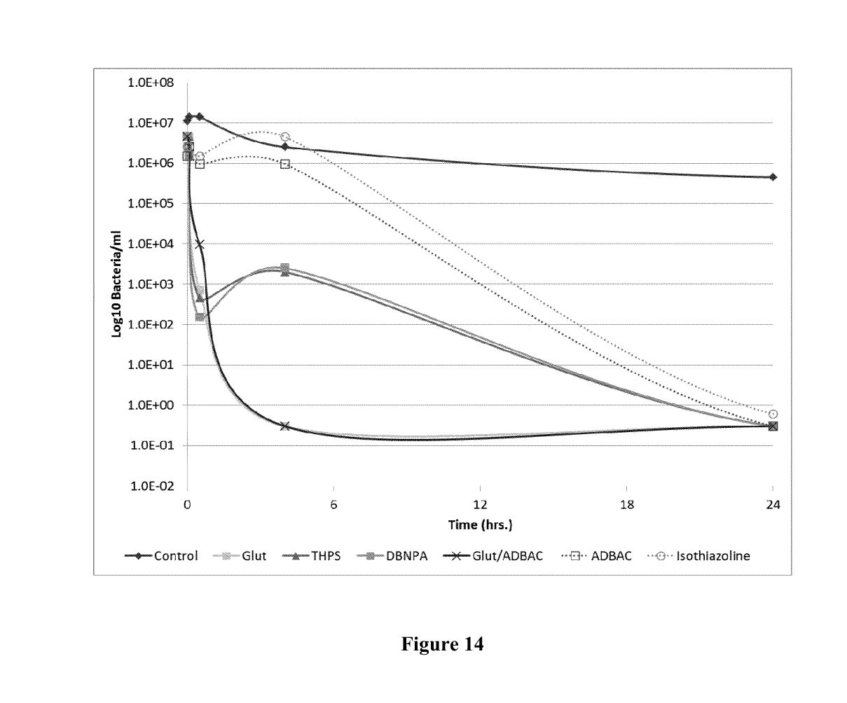

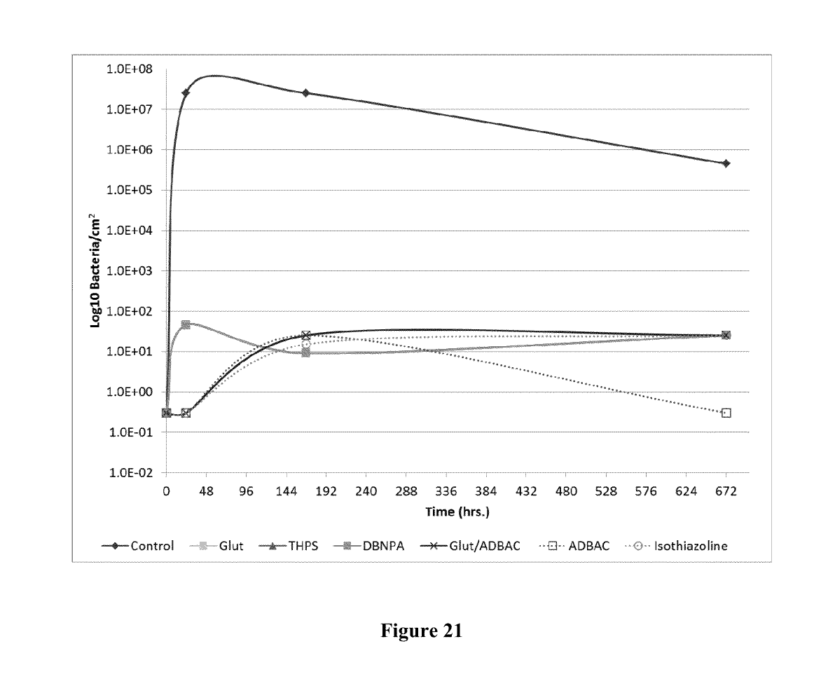

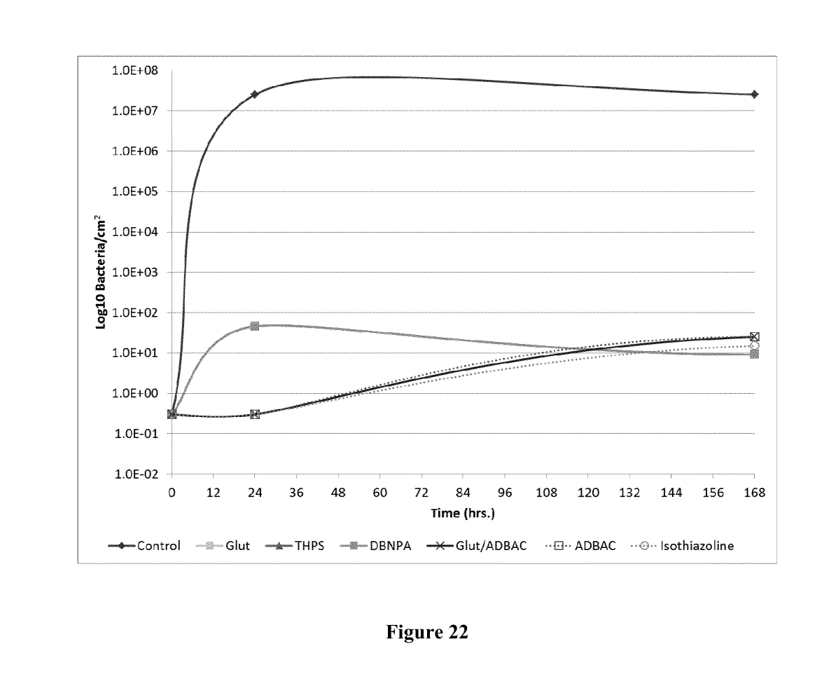

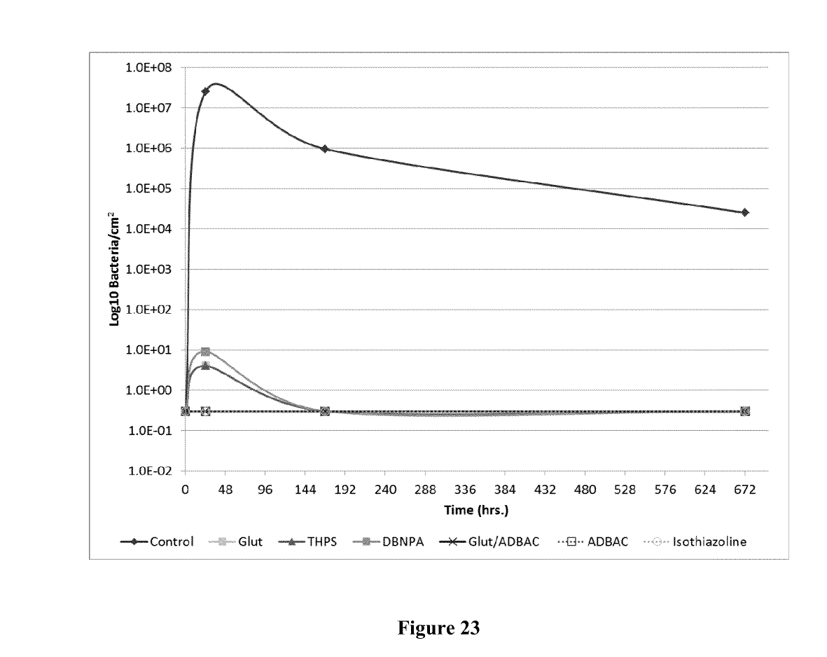

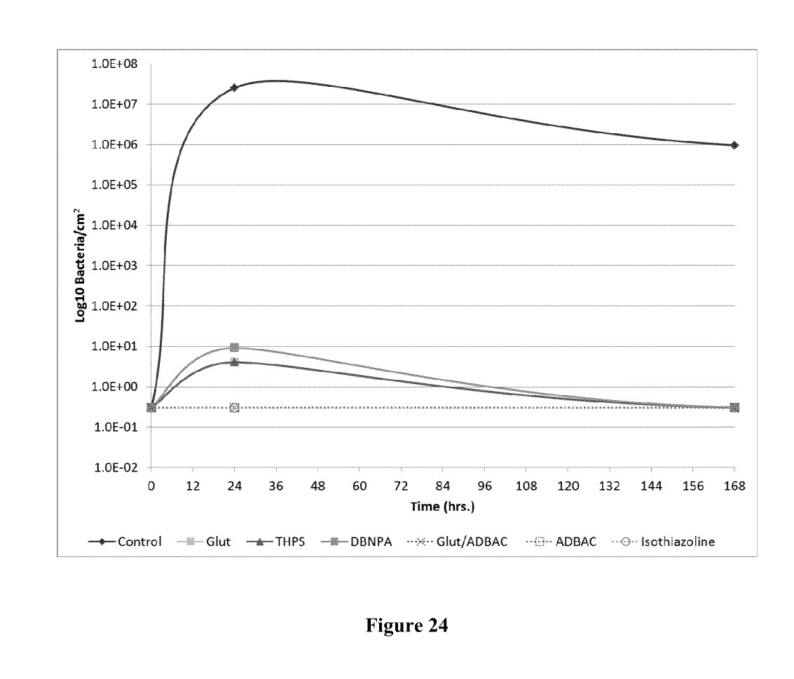

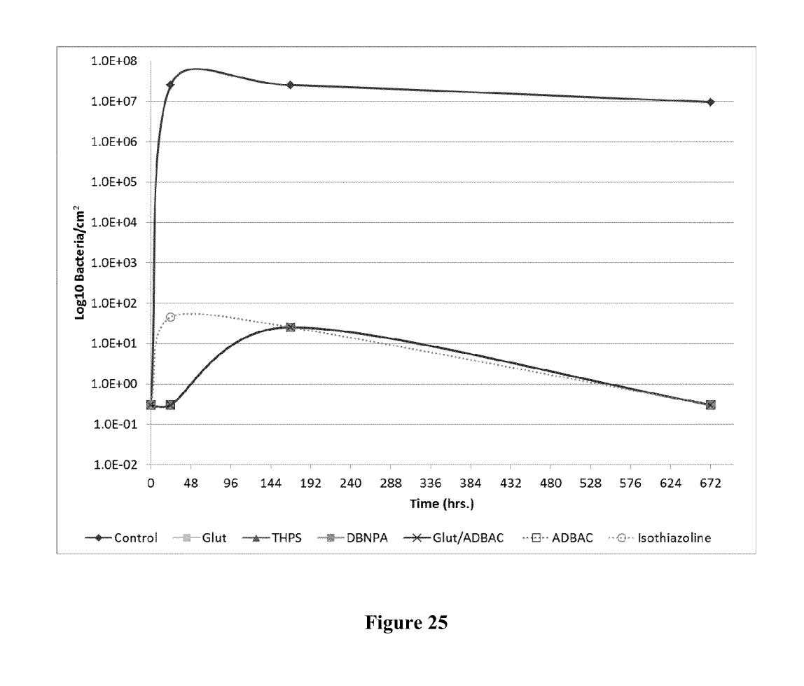

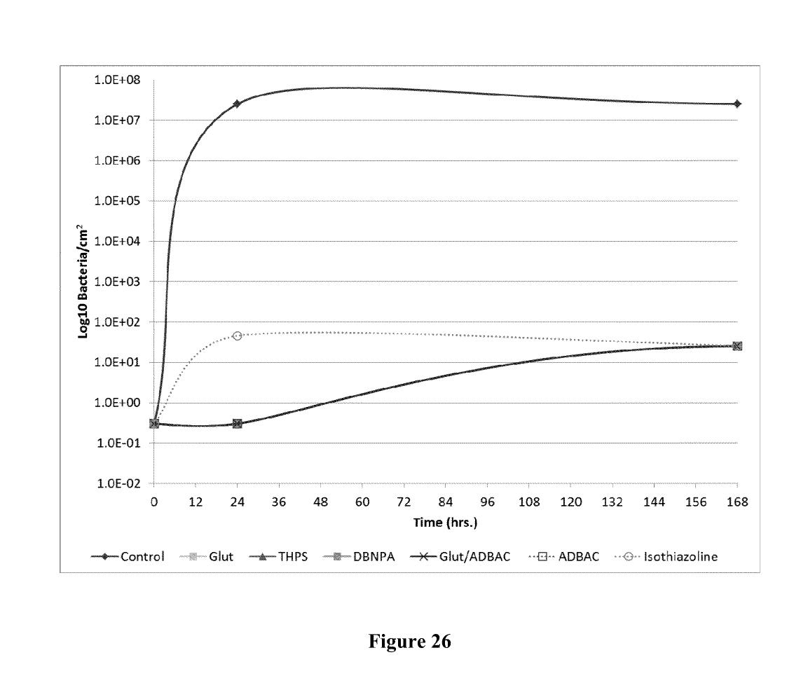

For all test samples, the surviving planktonic SRB, GHB, and APB were enumerated by the triplicate serial dilution method for MPN (most probable number) technique that is a method for viable bacteria enumeration at time points of 0 hours, 5 minutes, 30 minutes, 4 hours, 24 hours, 48 hours, 72 hours, 7 days, 14 days, and 28 days. Sessile samples were collected at 24 hours, 7 days, and 28 days, and the surviving planktonic and sessile bacteria were enumerated following the MPN method. The sessile evaluation were performed to establish if the biocide can not only kill the planktonic bacteria, but also what bacteria may drop out of solution, potentially affecting the planktonic data. All bacterial inoculations were performed according to NACE TMO194-04 recommendations for microbial monitoring in oilfield systems. The results are provided in the attached FIGS. 3-26.

Results

Planktonic Biocide Efficacy

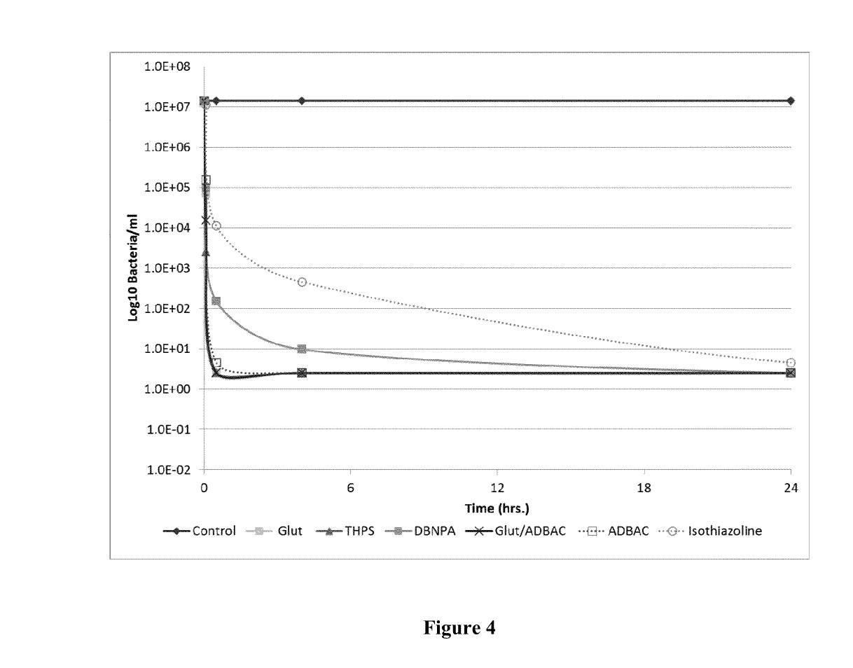

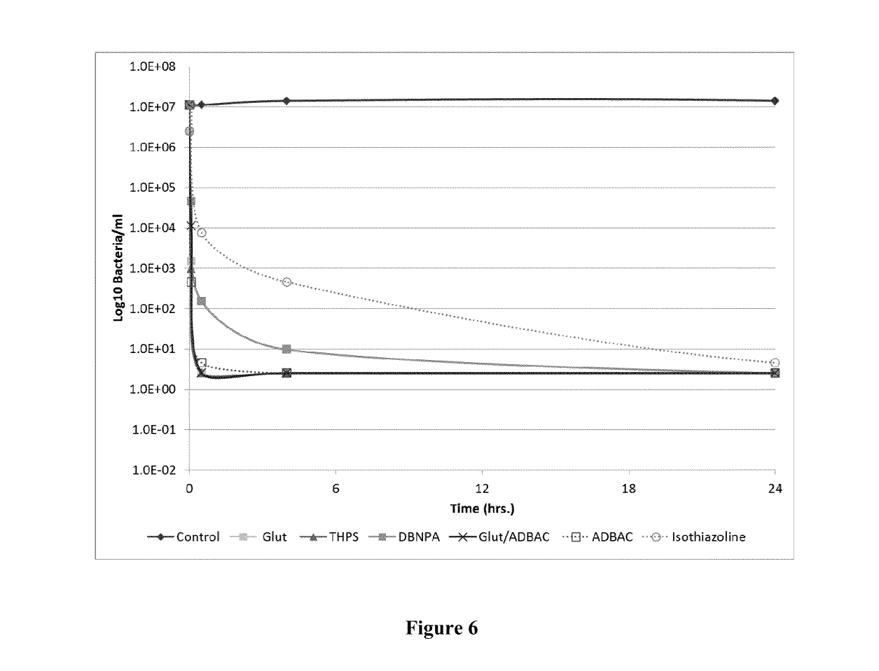

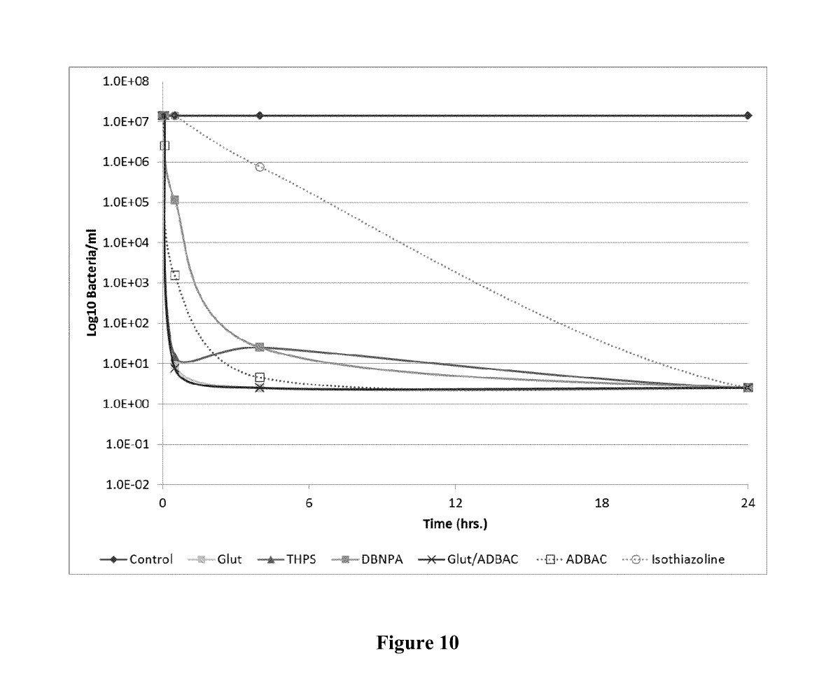

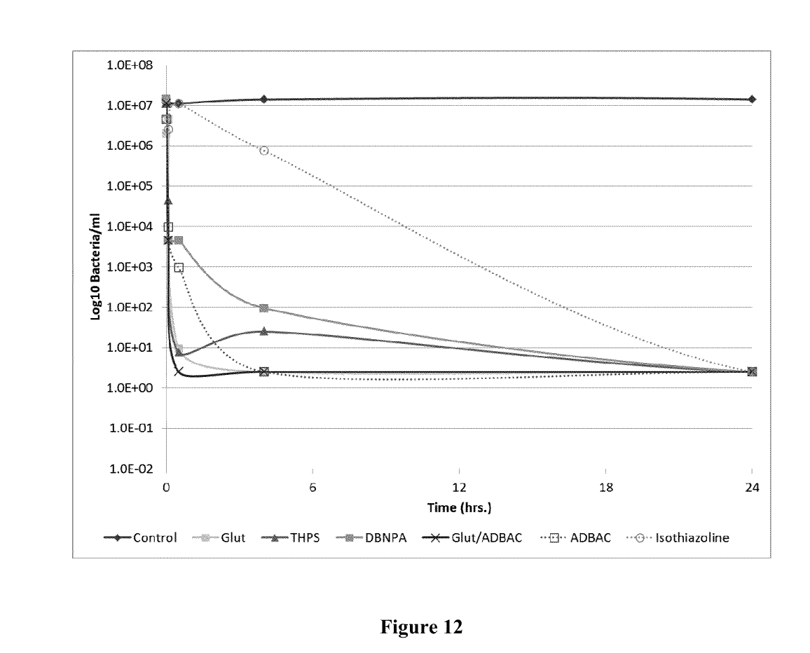

If you compare the first 4 hours of the correlated data sets, you can see a comparison of single-biocide systems (in the 4 hour sets, in which the AMA-324 is added after the 4-hour sample) and dual-biocide systems (in the 5 minute sets, in which the AMA-324 is added at 5 minutes). Comparing these data sets, there appears to be a synergistic effect with all first biocide components tested and AMA-324 product. For example, in comparison of FIG. 4 (5 minute test set) to FIG. 10 (4 hour test set), and FIG. 6 (5 minute test set) to FIG. 12 (4 hour test set), for the reactor test system for GHB and APB, the biocide efficacy rate is faster for the dual biocide treatment than the corresponding single (first biocide) treatment.

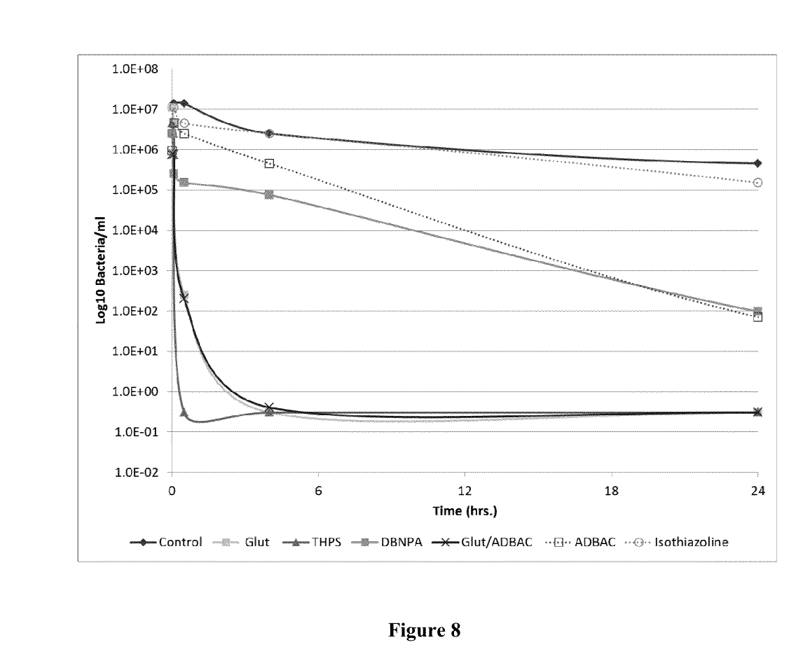

Similarly, when comparing FIG. 8 to FIG. 14, it appears that within the SRB biocide efficacy test, there is a synergistic effect with dual biocide systems using THPS, Glut, and Glut/ADBAC as the first biocidal component.

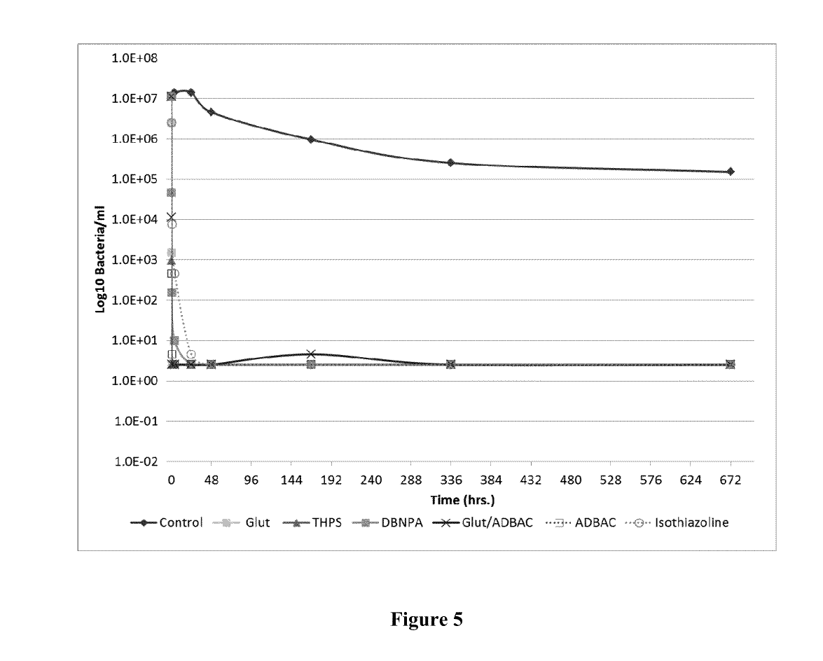

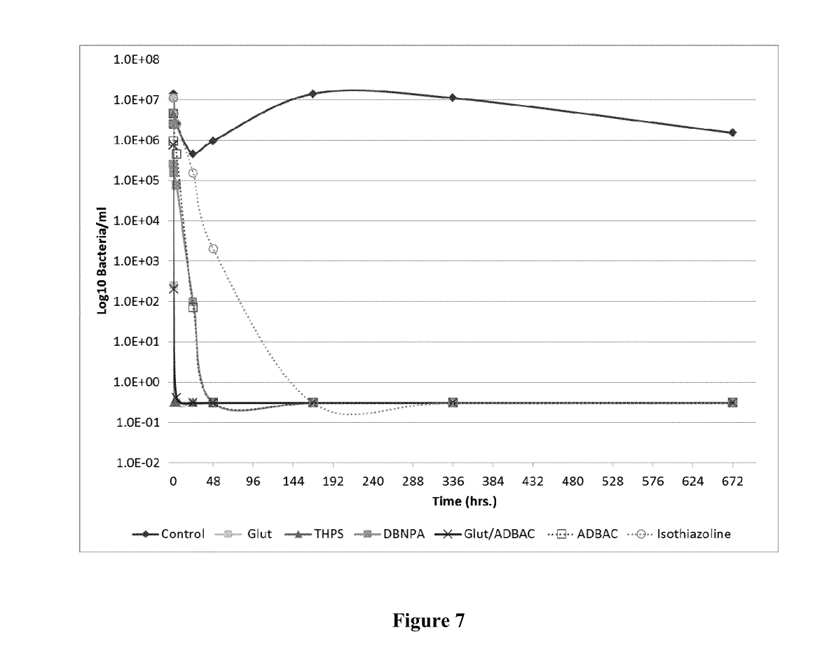

By looking at the biocidal efficacy at the 672 hrs. timepoint (28 days), it appears that data does not indicate any compatibility issue, because the first biocide components were still able to significantly reduce viable bacterial numbers within 4 hours. (FIGS. 3 & 9, FIGS. 5 & 11, and FIGS. 7 & 13)

The control data (no biocidal system) shows that there was no significant decrease in viability of GHB, APB, and SRB during the 28-day study, which indicates that any reduction in viable planktonic bacteria in the other samples was due to the action of the biocide(s). (FIGS. 3 & 9, FIGS. 5 & 11, and FIGS. 7 & 13)

Sessile Biocide Efficacy

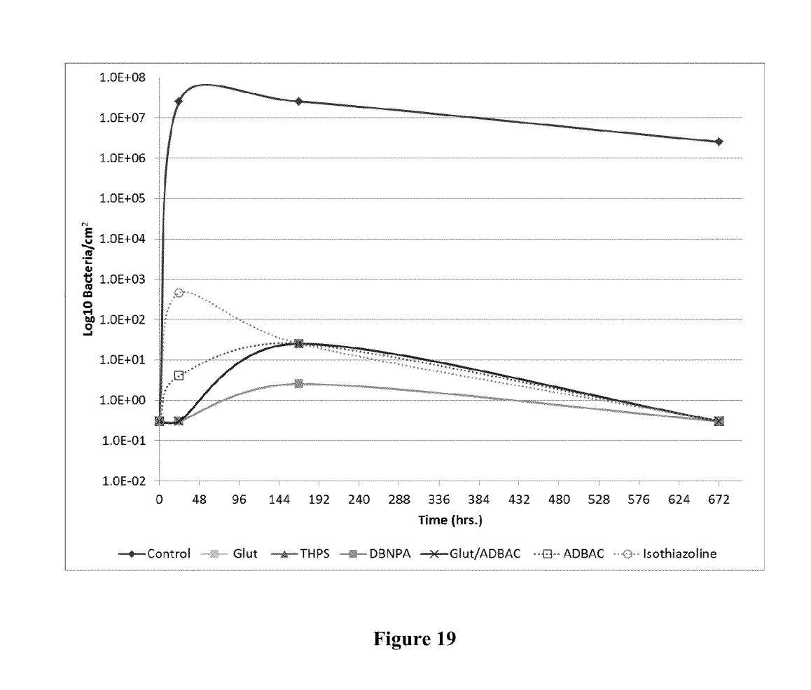

If you compare the first 4 hours of the correlated data sets, you can see a comparison of single-biocide systems (in the 4 hour sets, in which the AMA-324 is added after the 4-hour sample) and dual-biocide systems (in the 5 minute sets, in which the AMA-324 is added at 5 minutes). Comparing these data sets, for sessile SRBs, the addition of AMA-324 product resulted in a significantly faster biocidal rate. (For example, compare FIG. 19 to FIG. 25 and FIG. 20 to FIG. 26)

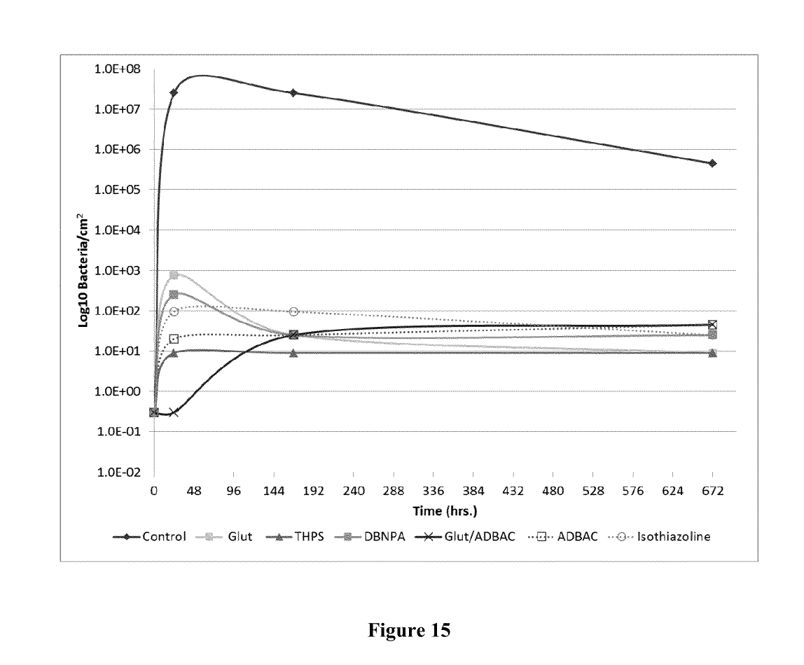

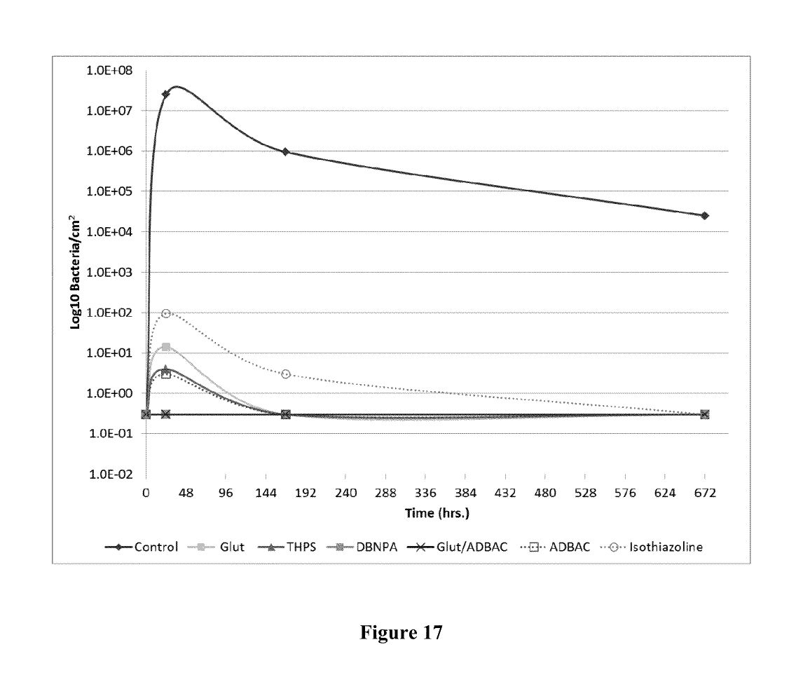

By looking at biocidal efficacy data at the 672 hr. timepoint (28 days) for both 5 min. test set and 4 hrs. test data, it does not appear that there is any compatibility issue between the biocides. The sessile biocidal efficacy for both test sets ended up about the same. (For example, compare FIG. 15 to FIG. 21, FIG. 17 to FIG. 23, and FIG. 19 to FIG. 25)

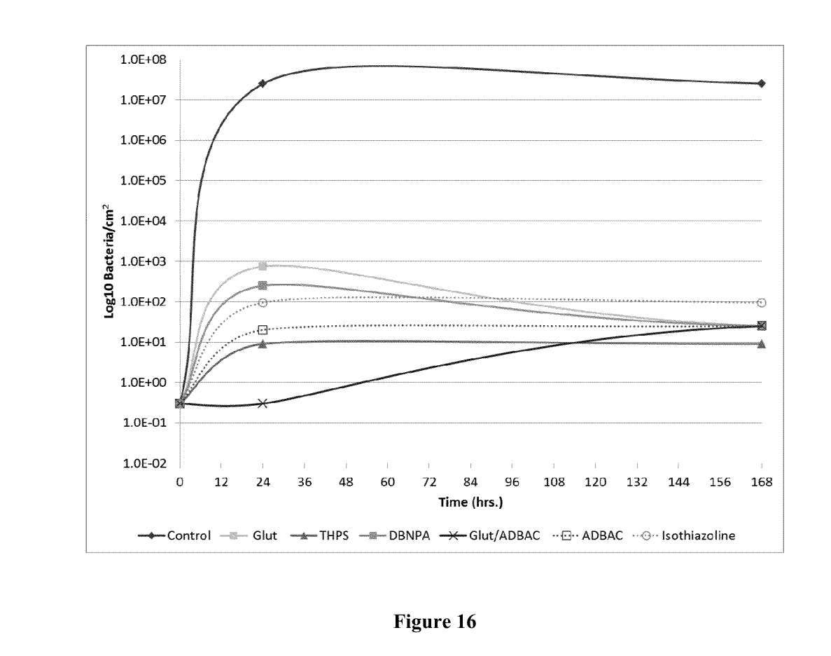

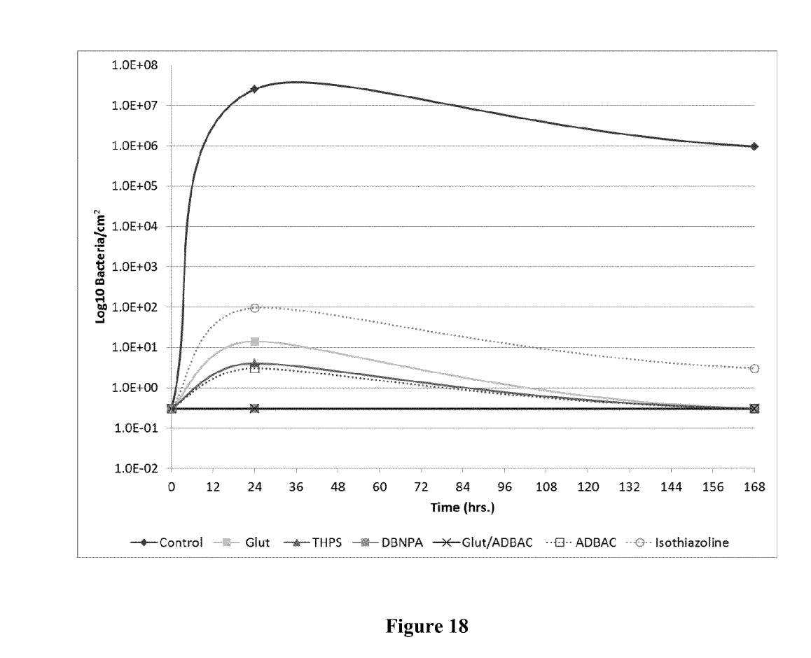

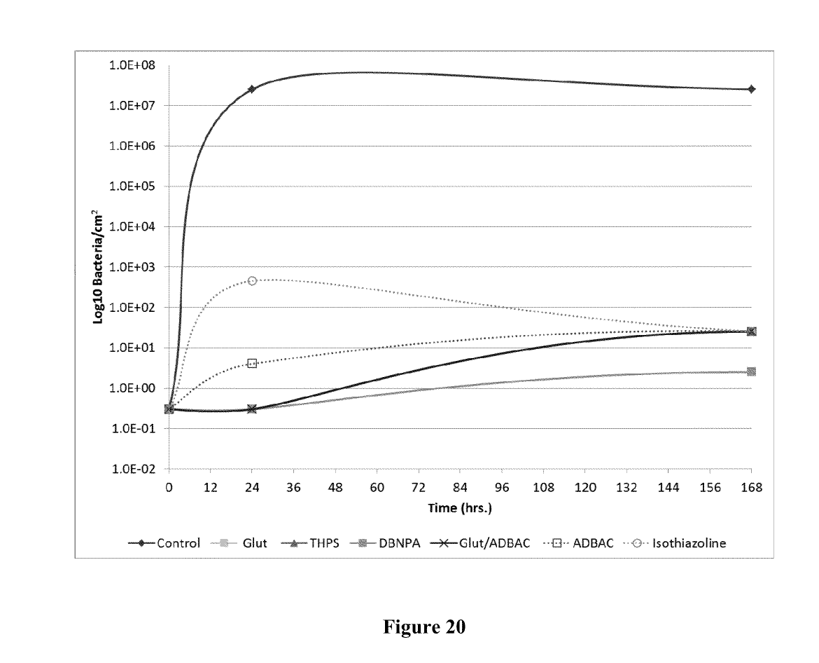

In most of the sessile biocide tests, there was a rapid development of the viable sessile population for all biocides tested (See FIGS. 16 & 22, FIGS. 18 & 24, and FIGS. 20 & 26), most probably due to planktonic bacteria settling out of the system, as a much closer inspection of the SRB data demonstrate the same phenomenon and SRB in a consortium system have doubling times of +20 hours2, thus a population flux to greater than 1×107 bacteria/cm² is technically not achievable simply through growth. (FIGS. 20 & 26)

### Example 4

### Biocide Efficacy Study in Hydrofracing Fluid

In this example, a biocidal system was evaluated in which the first biocidal component is chlorine dioxide, and the second biocide is AMA-324. The test was conducted in an active hydrofracing operation.

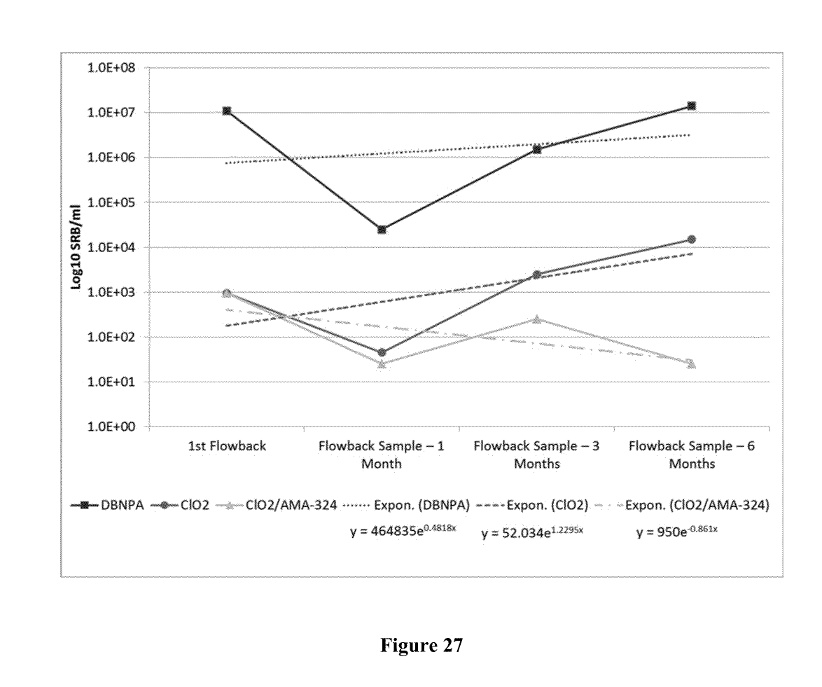

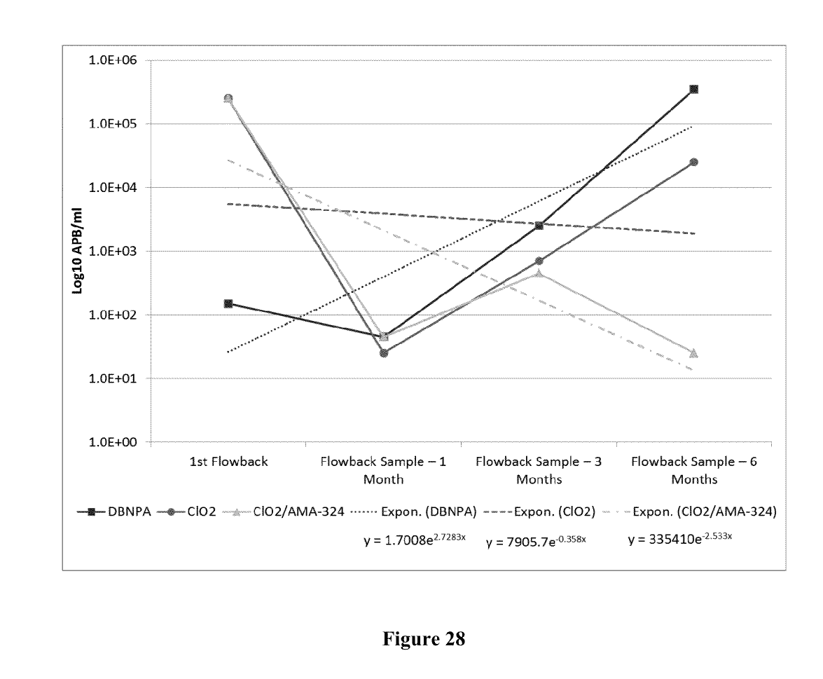

In the exemplary dual biocidal treatment, chlorine dioxide was injected into frac waters (brine containing from about 10,000-15,000 TDS) at a mixing manifold upstream of the frac tank, in an amount sufficient to produce 1 ppm residual ClO₂ in the frac waters as measured at the downstream blender. At the blender, AMA-324 was added to the frac waters to provide approximately 100 ppm (active) in the frac waters. For comparison, the frac waters were separately treated with single biocide treatment systems (DBNPA and ClO₂), which were injected only at the mixing manifold. In each test, the frac waters with the biocide(s) were injected into the formation in the normal course of operations. After injection, 2 L samples of flowback waters were extracted from the well head over time, The first samples (1^{st} Flowback in the Figures) was withdrawn 24 hours after drillout. The subsequent samples were withdrawn 1 month, 3 months, and 6 months thereafter. For each flowback sample, SRB and APB populations of the samples were enumerated. The results are shown in FIGS. 27 and 28.

We claim:

1. A method of treating a gas field fluid or oil field fluid comprises: a) adding a first biocide component to the gas field fluid or oil field fluid; and b) after a delay, adding the second biocide component to the gas field fluid or oil field fluid; wherein the delay is at least about 1 minute, wherein the first biocide component and second biocide component are added in an amount effective to control microbial growth or activity.

2. The method of claim 1, wherein the combined concentration of the active ingredients of the first biocide component and the second biocide component in the fluid is in the range of 5 ppm to 2000 ppm.

3. The method of claim 1, wherein the first biocide component comprises a biocide selected from the group consisting of glutaraldehyde, C₁₂₋₁₆-alkyl dimethyl benzyl ammonium chloride (ADBAC quat), glutaraldehyde and ADBAC quat, tetrakis(hydroxymethyl) phosphonium sulfate, 2,2-dibromo-3-nitrilopropionamide, [1,2-ethanediylbis(oxy)]bismethanol, 5-chloro-2-methyl-4-isothiazolin-3-one, chlorine dioxide and combinations thereof.

4. The method of claim 1, wherein the second biocide component is selected from a 3,5-dimethyl-1,3,5-thiadiazinane-2-thione and a monoalkyldithiocarbamate salt.

5. The method of claim 1, wherein the second biocide component is added in an amount effective to control microbial growth or activity in the fluid for a sustained period of time.

6. The method of claim 1, wherein the fluid is selected from a stimulation fluid, squeeze fluid, fracturing fluid, drilling mud, workover or completion fluid, hydrotest fluid, water injection or fluid injection for reservoir maintenance and Enhanced Oil Recovery (EOR).

7. The method of claim 1, wherein the fluid is an aqueous fluid or a fluid that comprises water.

8. The method of claim 1, wherein the fluid is a hydraulic fracturing fluid.

9. The method of claim 1, wherein the delay is about 5 minutes.

10. A method of treating a gas field fluid or oil field fluid, comprising: a) passing a gas field fluid or oil field fluid through a fluidic system; b) adding a first biocide component to the gas field fluid or oil field fluid via a first inlet to the fluidic system; and c) downstream from the first inlet, adding a second biocide component to the gas field fluid or oil field fluid via a second inlet to the fluidic system.

11. The method of claim 10, wherein the first biocide component is selected from the group consisting of glutaraldehyde, C₁₂₋₁₆-alkyl dimethyl benzyl ammonium chloride (ADBAC quat), glutaraldehyde and ADBAC quat, tetrakis(hydroxymethyl) phosphonium sulfate, 2,2-dibromo-3-nitrilopropionamide, [1,2-ethanediylbis(oxy)]bismethanol, 5-chloro-2-methyl-4-isothiazolin-3-one, chlorine dioxide and combinations thereof.

12. The method of claim 10, wherein the second biocide component comprises 3,5-dimethyl-1,3,5-thiadiazinane-2-thione.

13. The method of claim 10, wherein the second biocide component is added in an amount effective to control microbial growth and/or activity in the fluid for a sustained period of time.

14. The method of claim 11, wherein the fluid is selected from a stimulation fluid, squeeze fluid, hydraulic fracturing fluid, drilling mud, workover or completion fluid, hydrotest fluid, water injection or fluid injection for reservoir maintenance or Enhanced Oil Recovery (EOR).

15. The method of claim 10, wherein the fluid is an aqueous fluid or a fluid that comprises water.

16. The method of claim 10, wherein the fluid is a hydraulic fracturing fluid.

17. A treated gas field fluid or oil field fluid comprising a biocidal system comprising a first biocide component and a second biocide component wherein the first biocide and second biocide are present in an amount effective to control microbial activity.

18. The treated fluid of claim 17, wherein the first biocide component comprises a biocide selected from the group consisting of glutaraldehyde, C₁₂₋₁₆-alkyl dimethyl benzyl ammonium chloride (ADBAC quat), glutaraldehyde and ADBAC quat, tetrakis(hydroxymethyl) phosphonium sulfate, 2,2-dibromo-3-nitrilopropionamide, or [1,2-ethanediylbis(oxy)]bismethanol, 5-chloro-2-methyl-4-isothiazolin-3-one, chlorine dioxide and combinations thereof.

19. The treated fluid of claim 17, wherein the second biocide component is selected from 3,5-dimethyl-1,3,5-thiadiazinane-2-thione and a monoalkyldithiocarbamate salt.

20. The treated fluid of claim 17, wherein the fluid is a stimulation fluid, squeeze fluid, fracturing fluid, drilling mud, workover or completion fluid, hydrotest fluid, water injection or fluid injection for reservoir maintenance or Enhanced Oil Recovery (EOR).

21. The treated fluid of claim 17, wherein the fluid is an aqueous fluid or a fluid that comprises water.

22. The treated fluid of claim 17, wherein the fluid is a hydraulic fracturing fluid.

23. The treated fluid of claim 17, wherein the combined concentration of the active ingredients of the first biocide component and the second biocide component, as active ingredients, in the fluid is in the range of 5 ppm to 2000 ppm.

24. A biocidal system comprising a first biocide component and a second biocide component.

25. The biocidal system of claim 24, wherein the first biocide component comprises a biocide selected from the group consisting of glutaraldehyde, C₁₂₋₁₆-alkyl dimethyl benzyl ammonium chloride (ADBAC quat), glutaraldehyde and ADBAC quat, tetrakis(hydroxymethyl) phosphonium sulfate, 2,2-dibromo-3-nitrilopropionamide, [1,2-ethanediylbis(oxy)]bismethanol, 5-chloro-2-methyl-4-isothiazolin-3-one, chlorine dioxide and combinations thereof.

26. The biocidal system of claim 24, wherein the second biocide component is selected from a 3,5-dimethyl-1,3,5-thiadiazinane-2-thione and a monoalkyldithiocarbamate salt.
